(19) United States
(12) Patent Application Publication
(10) Pub. No.: US 2003/0144217 A1
(43) Pub. Date: Jul. 31, 2003

(54) Prodrugs of aspartyl protease inhibitors

(75) Inventors: Roger D. Tung, Beverly, MA (US); Michael R. Hale, Bedford, MA (US); Christopher T. Baker, Waltham, MA (US); Andrew Spaltenstein, Raleigh, NC (US); Eric S. Furfine, Durham, NC (US); Istvan Kaldor, Durham, NC (US); Wieslaw M. Kazmierski, Raleigh, NC (US)

Correspondence Address: FISH & NEAVE, 1251 AVENUE OF THE AMERICAS, 50TH FLOOR, NEW YORK, NY, 10020-1105, US

(73) Assignee: Vertex Pharmaceuticals Incorporated

(21) Appl. No.: 10/226,430

(22) Filed: Aug. 21, 2002

Related U.S. Application Data
(63) Continuation of application No. 09/998,617, filed on Nov. 30, 2001, now abandoned.

(51) Int. Cl. A61K 31/7024; A61K 31/66; A61K 31/496; A61K 31/34; A61K 31/44; A61K 31/4172; A61K 31/221

(52) U.S. Cl. 514/23; 514/114; 514/220; 514/252.12; 514/423; 514/254.01; 514/473; 514/357; 514/467; 514/547; 514/550; 514/551; 536/18.7; 544/388; 546/329; 548/530; 558/166; 549/430; 549/478

(57) ABSTRACT

The present invention relates to prodrugs of a class of sulfonamides which are aspartyl protease inhibitors. In one embodiment, this invention relates to a novel class of prodrugs of HIV aspartyl protease inhibitors characterized by favorable aqueous solubility, high oral bioavailability and facile in vivo generation of the active ingredient. This invention also relates to pharmaceutical compositions comprising these prodrugs. The prodrugs and pharmaceutical compositions of this invention are particularly well suited for decreasing the pill burden and increasing patient compliance. This invention also relates to methods of treating mammals with these prodrugs and pharmaceutical compositions.

### TECHNICAL FIELD OF THE INVENTION

The present invention relates to prodrugs of a class of sulfonamides which are aspartyl protease inhibitors. In one embodiment, this invention relates to a novel class of prodrugs of HIV aspartyl protease inhibitors characterized by favorable aqueous solubility, high oral bioavailability and facile in vivo generation of the active ingredient. This invention also relates to pharmaceutical compositions comprising these prodrugs. The prodrugs and pharmaceutical compositions of this invention are particularly well suited for decreasing the pill burden and increasing patient compliance. This invention also relates to methods of treating mammals with these prodrugs and pharmaceutical compositions.

### BACKGROUND OF THE INVENTION

Aspartyl protease inhibitors are considered the most effective current drug in the fight against HIV infection. These inhibitors, however, require certain physicochemical properties in order to achieve good potency against the enzyme. One of these properties is high hydrophobicity. Unfortunately, this property results in poor aqueous solubility and low oral bioavailability.

U.S. Pat. No. 5,585,397 describes a class of sulfonamide compounds that are inhibitors of the aspartyl protease enzyme. These compounds illustrate the drawbacks concomitant to pharmaceutical compositions comprising hydrophobic aspartyl protese inhibitors. For example, VX-478 (4-amino-N-((2-syn,3S)-2-hydroxy-4-phenyl-2((S)-tetrahydrofuran-3-yl-oxycarbonylamino)-butyl-N-isobutyl-benzenesulfonamide) is an aspartyl protease inhibitor disclosed in the '397 patent. In its mesylate salt form, VX-478 has a relatively low aqueous solubility. While the oral bioavailability of this inhibitor in a “solution” formulation is excellent, the dosage of VX-478 in this form is severely limited by the amount of liquid present in the particular liquid dosage from, e.g., encapsulated into a soft gelatin capsule. A higher aqueous solubility would increase drug load per unit dosage of VX-478.

Currently, the mesylate formulation of VX-478 produces an upper limit of 150 mg of VX-478 in each capsule. Given a therapeutic dose of 2400 mg/day of VX-478, this formulation would require a patient to consume 16 capsules per day. Such a high pill burden would likely result in poor patient compliance, thus producing sub-optimal therapeutic benefit of the drug. The high pill burden is also a deterrent to increasing the amount of the drug administered per day to a patient. Another drawback of the pill burden and the concomitant patient compliance problem is in the treatment of children infected with HIV.

Furthermore, these “solution” formulations, such as the mesylate fomulation, are at a saturation solubility of VX-478. This creates the real potential of having the drug crystallize out of solution under various storage and/or shipping conditions. This, in turn, would likely result in a loss of some of the oral bioavailability achieved with VX-478.

One way of overcoming these problems is to develop a standard solid dosage form, such as a tablet or a capsule or a suspension form. Unfortunately, such solid dosage forms have much lower oral bioavailability of the drug.

Thus, there is a need to improve the drug load per unit dosage form for aspartyl protease inhibitors. Such an improved dosage form would reduce the pill burden and increase patient compliance. It would also provide for the possibility of increasing the amounts of the drug administered per day to a patient.

### SUMMARY OF THE INVENTION

The present invention provides novel prodrugs of a class of sulfonamide compounds that are inhibitors of aspartyl protease, in particular, HIV aspartyl protease. These prodrugs are characterized by high aqueous solubility, increased bioavailability and are readily matabolized into the active inhibitors in vivo. The present invention also provides pharmaceutical compositions comprising these prodrugs and methods of treating HIV infection in mammals using these prodrugs and the pharmaceutical compositions thereof.

These prodrugs can be used alone or in combination with other therapeutic or prophylactic agents, such as anti-virals, antibiotics, immunomodulators or vaccines, for the treatment or prophylaxis of viral infection.

It is a principal object of this invention to provide novel prodrugs of a class of sulfonamides which are aspartyl protease inhibitors, and particularly, HIV aspartyl protease inhibitors. This novel class of sulfonamides is represented by formula I:

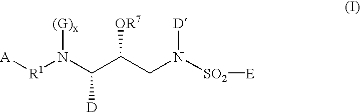

wherein:

each R¹ is independently selected from the group consisting of C(O)—, —S(O)₂—, —C(O)—C(O)—, —O—C(O)—, —O—S(O)₂, —NR²—S(O)₂—, —NR²—C(O)— and —NR²—C(O)—C(O)—;

each A is independently selected from the group consisting of 5-7 membered monocyclic heterocycles containing from 1-3 endocyclic heteroatoms, which may be optionally methylated at the point of attachment, optionally benzofused, optionally attached through a C₁-C₃ alkyl linker and optionally fused with a 5-7 membered monocyclic heterocycle containing from 1-2 endocyclic heteroatoms, and wherein unmethylated THF is expressly excluded;

each Ht is independently selected from C₃-C₇ cycloalkyl; C₅-C₇ cycloalkenyl; C₆-C₁₀ aryl; or a 5-7 membered saturated or unsaturated heterocycle, containing one or more heteroatoms selected from N, N(R²), O, S and S(O)ₙ; wherein said aryl or said heterocycle is optionally fused to Q; and wherein any member of said Ht is optionally substituted with one or more substituents independently selected from oxo, —OR², SR², —R², —N(R²)(R²), —R²—OH, —CN, —CO₂R², —C(O)—N(R²)₂, —S(O)₂—N(R²)₂, —N(R²)—C(O)—R², —C(O)—R², —S(O)ₙ—R², —OCF₃, —S(O)ₙ—Q, methylenedioxy, —N(R²)—S(O)₂(R²), halo, —CF₃, —NO₂, Q, —OQ, —OR⁷, —SR⁷, —R⁷, —N(R²)(R⁷) or —N(R⁷)₂;

each Q is independently selected from a 3-7 membered saturated, partially saturated or unsaturated carbocyclic ring system; or a 5-7 membered saturated, partially saturated or unsaturated heterocyclic ring containing one or more heteroatoms selected from O, N, S, S(O)ₙ or N(R²); wherein Q is optionally substituted with one or more groups selected from oxo, —OR², —R², —N(R²)₂, —N(R²)—C(O)—R², —R²—OH, —CN, —CO₂R², —C(O)—N(R²)₂, halo or —CF₃;

each R² is independently selected from the group consisting of H and C₁-C₃ alkyl optionally substituted with Q;

each x is independently 0 or 1;

each R³ is independently selected from the group consisting of H, Ht, C₁-C₆ alkyl and C₂-C₆ alkenyl wherein any member of said R³, except H, may be optionally substituted with one or more substituents selected from the group consisting of —OR², —C(O)—NH—R², —S(O)ₙ—N(R²)(R²), Ht, —CN, —SR², —CO₂R², NR²—C(O)—R²;

each n is independently 1 or 2;

G, when present, is selected from H, R⁷ or C₁-C₄ alkyl, or, when G is C₁-C₄ alkyl, G and R⁷ are bound to one another either directly or through a C₁-C₃ linker to form a heterocyclic ring; or

when G is not present (i.e., when x in (G)ₓ is 0), then the nitrogen to which G is attached is bound directly to the R⁷ group on —OR⁷;

each D and D′ is independently selected from the group consisting of Q; C₁-C₅ alkyl, which may be optionally substituted with one or more groups selected from C₃-C₆ cycloalkyl, —OR², —R³, —O—Q, —S—Q and Q; C₂-C₄ alkenyl, which may be optionally substituted with one or more groups selected from the group consisting of C₃-C₆ cycloalkyl, —OR², R³, O—Q and Q; C₃-C₆ cycloalkyl, which may be optionally substituted with or fused with Q; and C₅-C₆ cycloalkenyl, which may be optionally substituted with or fused with R⁶;

each E is independently selected from the group consisting of Ht; —O—Ht; Ht—Ht; —O—R³; —NR²R³; C₁-C₆ alkyl, which may be optionally substituted with one or more groups selected from the group consisting of R⁴ and Ht; and C₂-C₆ alkenyl, which may be optionally substituted with one or more groups selected from the group consisting of R⁴ and Ht; C₃-C₆ saturated carbocycle, which is optionally substituted with one or more groups selected from R⁴ or Ht; or C₅-C₆ unsaturated carbocycle, which is optionally substituted with one or more groups selected from R⁴ or Ht;

each R⁴ is independently selected from the group consisting of OR², —C(O)—NHR², S(O)₂—NHR², halo, NR²—C(O)—R² and —CN;

each R⁵ is independently selected from the group consisting of H and C₁-C₄ alkyl optionally substituted with aryl; and

each R⁶ is independently selected from the group consisting of aryl, carbocycle and heterocycle, wherein said aryl, carbocycle or heterocycle may be optionally substituted with one or more groups selected from the group consisting of oxo, —OR⁵, —R⁵, N(R⁵)(R⁵), N(R⁵)—C(O)—R⁵, —R⁵—OH, —CN, CO₂R⁵, C(O)—N(R⁵)(R⁵), halo and CF₃;

each R⁷ is independently selected from

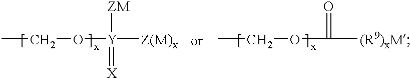

wherein each M is independently selected from H, Li, Na, K, Mg, Ca, Ba, —N(R²)₄, C₁-C₁₂-alkyl, C₂-C₁₂-alkenyl, —R⁶; wherein 1 to 4 —CH₂ radicals of the alkyl or alkenyl group, other than the —CH₂ that is bound to Z, is optionally replaced by a heteroatom group selected from O, S, S(O), S(O₂), or N(R²); and wherein any hydrogen in said alkyl, alkenyl or R⁶ is optionally replaced with a substituent selected from oxo, —OR², —R², N(R²)₂, N(R²)₃, R²OH, —CN, —CO₂R², —C(O)—N(R²)₂, S(O)₂—N(R²)₂, N(R²)—C(O)—R₂, C(O)R², —S(O)ₙ—R², OCF₃, —S(O)ₙ—R⁶, N(R²)—S(O)₂(R²), halo, —CF₃, or —NO₂;

M′ is H, C₁-C₁₂-alkyl, C₂-C₁₂-alkenyl, —R⁶; wherein 1 to 4 —CH₂⁻ radicals of the alkyl or alkenyl group is optionally replaced by a heteroatom group selected from O, S, S(O), S(O₂), or N(R²); and wherein any hydrogen in said alkyl, alkenyl or R⁶ is optionally replaced with a substituent selected from oxo, —OR², —R², —N(R²)₂, N(R²)₃, —R²OH, —CN, —CO₂R², —C(O)—N(R²)₂, —S(O)₂—N(R²)₂, —N(R²)—C(O)—R₂, —C(O)R², —S(O)ₙ—R², —OCF₃, —S(O)ₙ—R⁶, —N(R²)—S(O)₂(R²), halo, —CF₃, or —NO₂;

Z is O, S, N(R²)₂, or, when M is absent, H;

Y is P or S;

X is O or S; and

R⁹ is C(R²)₂, O or N(R²); and wherein when Y is S, Z is not S; and

R⁶ is a 5-6 membered saturated, partially saturated or unsaturated carbocyclic or heterocyclic ring system, or an 8-10 membered saturated, partially saturated or unsaturated bicyclic ring system; wherein any of said heterocyclic ring systems contains one or more heteroatoms selected from O, N, S, S(O)ₙ or N(R²); and wherein any of said ring systems optionally contains 1 to 4 substituents independently selected from OH, C₁-C₄ alkyl, O—C₁-C₄ alkyl or OC(O)C₁-C₄ alkyl.

It is also an object of this invention to provide pharmaceutical compositions comprising the sulfonamides of formula I and methods for their use as inhibitors of HIV aspartyl protease.

### DETAILED DESCRIPTION OF THE INVENTION

In order that the invention herein described may be more fully understood, the following detailed description is set forth. In the description, the following abbreviations are used:

| Designation | Reagent or Fragment |
|---|---|
| Ac | acetyl |
| Me | methyl |
| Et | ethyl |
| Bn | benzyl |
| Trityl | triphenylmethyl |
| Asn | D- or L-asparagine |
| Ile | D- or L-isoleucine |
| Phe | D- or L-phenylalanine |
| Val | D- or L-valine |
| Boc | tert-butoxycarbonyl |
| Cbz | benzyloxycarbonyl (carbobenzyloxy) |
| Fmoc | 9-fluorenylmethoxycarbonyl |
| DCC | dicyclohexylcarbodiimide |
| DIC | diisopropylcarbodiimide |
| EDC | 1-(3-dimethylaminopropyl)-3- |
| | ethylcarbodiimide hydrochloride |
| HOBt | 1-hydroxybenzotriazole |
| HOSu | 1-hydroxysuccinimide |
| TFA | trifluoroacetic acid |
| DIEA | diisopropylethylamine |
| DBU | 1,8-diazabicyclo(5.4.0)undec-7-ene |
| EtOAc | ethyl acetate |
| t-Bu | tert-butyl |
| iBu | iso-butyl |
| DMF | dimethylformamide |
| THP | tertrahydropyran |
| THF | tetrahydrofuran |
| TMSC1 | chlorotrimethylsilane |
| DMSO | dimethylsulfoxide |

The following terms are employed herein:

Unless expressly stated to the contrary, the terms “SO₂—” and “S(O)₂—” as used herein refer to a sulfone or sulfone derivative (i.e., both appended groups linked to the S), and not a sulfinate ester.

The term “backbone” refers to the structural representation of a compound of this invention, as set forth in the figures drawn in this application.

For the compounds of formula I, and intermediates thereof, the stereochemistry of the —OR⁷ group is defined relative to D on the adjacent carbon atom, when the molecule is drawn in an extended zig-zag representation (such as that drawn for compounds of formula X, XI, XII, XIII, XX, XXI, and XXII). If both —OR⁷ and D reside on the same side of the plane defined by the extended backbone of the compound, the stereochemistry of the —OR⁷ bearing carbon atom will be referred to as “syn”. If —OR⁷ and D reside on opposite sides of that plane, the stereochemistry of the —OR⁷ bearing carbon atom will be referred to as “anti”.

As used herein, the term “alkyl”, alone or in combination with any other term, refers to a straight-chain or branch-chain saturated aliphatic hydrocarbon radical containing the specified number of carbon atoms, or where no number is specified, preferably from 1-10 and more preferably from 1-5 carbon atoms. Examples of alkyl radicals include, but are not limited to, methyl, ethyl, n-propyl, isopropyl, n-butyl, isobutyl, sec-butyl, tert-butyl, pentyl, isoamyl, n-hexyl and the like.

The term “alkenyl”, alone or in combination with any other term, refers to a straight-chain or branched-chain mono- or poly-unsaturated aliphatic hydrocarbon radical containing the specified number of carbon atoms, or where no number is specified, preferably from 2-10 carbon atoms and more preferably, from 2-6 carbon atoms. Examples of alkenyl radicals include, but are not limited to, ethenyl, E- and Z-propenyl, isopropenyl, E- and Z-butenyl, E- and Z-isobutenyl, E- and Z-pentenyl, E- and Z-hexenyl, E,E-, E,Z-, Z,E- and Z,Z-hexadienyl and the like.

The term “aryl”, alone or in combination with any other term, refers to a carbocyclic aromatic radical (such as phenyl or naphthyl) containing the specified number of carbon atoms, preferably from 6-14 carbon atoms, and more preferably from 6-10 carbon atoms. Examples of aryl radicals include, but are not limited to phenyl, naphthyl, indenyl, indanyl, azulenyl, fluorenyl, anthracenyl and the like.

The term “cycloalkyl”, alone or in combination with any other term, refers to a cyclic saturated hydrocarbon radical containing the specified number of carbon atoms, preferably from 3-7 carbon atoms. Examples of cycloalkyl radicals include, but are not limited to, cyclopropyl, cyclobutyl, cyclopentyl, cyclohexyl, cycloheptyl and the like.

The term “cycloalkenyl”, alone or in combination with any other term, refers to a cyclic hydrocarbon radical containing the specified number of carbon atoms with at least one endocyclic carbon-carbon bond. Where no number of carbon atoms is specified, a cycloalkenyl radical preferably has from 5-7 carbon atoms. Examples of cycloalkenyl radicals include, but are not limited to, cyclopentenyl, cyclohexenyl, cyclopentadienyl and the like.

The term “THF” refers to a tetrahydrofuran ring attached at any ring carbon resulting in a stable structure.

The term “carbocycle” refers to a stable nonaromatic 3 to 8-membered carbon ring which may be saturated, mono-unsaturated or poly-unsaturated. The carbocycle may be attached at any endocyclic carbon atom which results in a stable structure. Preferred carbocycles have 5-6 carbons.

The term “heterocycle”, unless otherwise defined herein, refers to a stable 3-7 membered monocyclic heterocyclic ring or 8-11 membered bicyclic heterocyclic ring which is either saturated or unsaturated, and which may be optionally benzofused if monocyclic. Each heterocycle consists of one or more carbon atoms and from one to four heteroatoms selected from the group consisting of nitrogen, oxygen and sulfur. As used herein, the terms “nitrogen and sulfur heteroatoms” include any oxidized form of nitrogen and sulfur, and the quaternized form of any basic nitrogen. In addition, any ring nitrogen may be optionally substituted with a substituent R², as defined herein for compounds of formula I. A heterocycle may be attached at any endocyclic carbon or heteroatom which results in the creation of a stable structure. A heterocycle may be attached at any endocyclic carbon or heteroatom which results in the creation of a stable structure. Preferred heterocycles include 5-7 membered monocyclic heterocycles and 8-10 membered bicyclic heterocycles. Preferred heterocycles defined above include, for example, benzimidazolyl, imidazolyl, imidazolinoyl, imidazolidinyl, quinolyl, isoquinolyl, indolyl, indazolyl, indazolinolyl, perhydropyridazyl, pyridazyl, pyridyl, pyrrolyl, pyrrolinyl, pyrrolidinyl, pyrazolyl, pyrazinyl, quinoxolyl, piperidinyl, pyranyl, pyrazolinyl, piperazinyl, pyrimidinyl, pyridazinyl, morpholinyl, thiamorpholinyl, furyl, thienyl, triazolyl, thiazolyl, β-carbolinyl, tetrazolyl, thiazolidinyl, benzofuranoyl, thiamorpholinyl sulfone, oxazolyl, benzoxazolyl, oxopiperidinyl, oxopyrroldinyl, oxoazepinyl, azepinyl, isoxazolyl, isothiazolyl, furazanyl, tetrahydropyranyl, tetrahydrofuranyl, thiazolyl, thiadiazoyl, dioxolyl, dioxinyl, oxathiolyl, benzodioxolyl, dithiolyl, thiophenyl, tetrahydrothiophenyl and sulfolanyl.

The term “halo” refers to a radical of fluorine, chlorine, bromine or iodine.

The term “linker” refers to a structural unit through which two other moieties are joined. For example, the term “C₁-C₃ alkyl linker” refers to a 1-3 carbon unit which attaches two other moieties together.

The terms “oxygenated heterocycle” and “heterocycle containing endocyclic oxygen atoms” are used interchangeably and refer to a monocyclic or bicyclic heterocycle containing a specified number of endocyclic oxygen atoms. Preferably, such oxygenated heterocycles contain only endocyclic oxygen heteroatoms. Examples of oxygenated heterocycles, include, but are not limited to: dioxanyl, dioxolanyl, tetrahydrofuranyl, tetrahydrofurodihydrofuranyl, tetrahydropyranyl, tetrahydropyranodihydrofuranyl, dihydropyranyl, tetrahydrofurofuranyl and tetrahydropyranofuranyl.

The terms “HIV protease” and “HIV aspartyl protease” are used interchangeably and refer to the aspartyl protease encoded by the human immunodeficiency virus type 1 or 2. In a preferred embodiment of this invention, these terms refer to the human immunodeficiency virus type 1 aspartyl protease.

The term “antiviral agent” or “anti-retroviral agent” refers to a compound or drug which possesses viral inhibitory activity. Such agents include reverse transcriptase inhibitors (including nucleoside and non-nucleoside analogs) and protease inhibitors. Preferably the protease inhibitor is an HIV protease inhibitor. Examples of nucleoside analog reverse transcriptase inhibitors include, but are not limited to, zidovudine (AZT), dideoxycytidine (ddC), didanosine (ddI), stavudine (d4T), 3TC, 935U83, 1592U89 and 524W91. Examples of non-nucleoside analog reverse transcriptase inhibitors include, but are not limited to delavirdine (U90) and nevirapine. Examples of HIV protease inhibitors include, but are not limited to, saquinavir (Ro 318959), L-735,524, ABT 538 (A80538), AG 1343, XM 412, XM 450, BMS 186318 and CPG 53,437.

The term “pharmaceutically effective amount” refers to an amount effective in treating HIV infection in a patient either as monotherapy or in combination with other agents. The term “treating” as used herein refers to the alleviation of symptoms of a particular disorder in a patient or the improvement of an ascertainable measurement associated with a particular disorder. Specifically, with respect to HIV, effective treatment using the compounds and compositions of this invention would result in an improvement in an HIV associated ascertainable measurement. Such measurements include, but are not limited to, reduction in viral load in plasma or another defined tissue compartment as measured by, e.g. RT-PCR or branched-chain DNA PCR or culturable virus measurements, □-2 microglobulin or p24 levels, number of CD₄⁺ cells or ratio of CD₄⁺/CD₈⁺ cells, or functional markers such as improvement in quality of life or ability to carry out normal functions or reduction in immunosuppression-related effects. The term “prophylactically effective amount” refers to an amount effective in preventing HIV infection in a patient. As used herein, the term “patient” refers to a mammal, including a human.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the antiretroviral agent.

The term “point of attachment” refers to the atom through which a moiety is attached to a specified structure. When a point of attachment may be optionally methylated, the point of attachment is the carbon atom through which a moiety is attached to a specified structure.

The term “substituted”, whether express or implied and whether preceded by the term “optionally” or not, refers to the replacement of one or more hydrogen radicals in a given structure with the radical of a specified substituent. When more than one position in a given structure may be substituted with a substituent selected from a specified group, the substituents may be either the same or different at every position. Typically, when a structure may be optionally substituted, 0-3 substitutions are preferred, and 0-1 substitution is most preferred. Most preferred substituents are those which enhance protease inhibitory activity or intracellular antiviral activity in permissive mammalian cells or immortalized mammalian cell lines, or which enhance deliverability by enhancing solubility characteristics or enhancing pharmacokinetic or pharmacodynamic profiles as compared to the unsubstituted compound. Other most preferred substituents include those used in the compounds shown in Table I.

Pharmaceutically acceptable salts of the compounds of this invention include those derived from pharmaceutically acceptable inorganic and organic acids and bases. Examples of suitable acids include hydrochloric, hydrobromic, sulfuric, nitric, perchloric, fumaric, maleic, phosphoric, glycollic, lactic, salicylic, succinic, p-toluenesulfonic, tartaric, acetic, citric, methanesulfonic, ethanesulfonic, formic, benzoic, malonic, naphthalene-2-sulfonic and benzenesulfonic acids. Preferred acids include hydrochloric, sulfuric, methanesulfonic and ethanesulfonic acids. Methanesulfonic acid is most preferred. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium), alkaline earth metal (e.g., magnesium), ammonium and N—(C₁₋₄ alkyl)₄⁺ salts.

The term “thiocarbamates” refers to compounds containing the functional group N—SO₂—O.

The compounds of this invention contain one or more asymmetric carbon atoms and thus occur as racemates and racemic mixtures, single enantiomers, diastereomeric mixtures and individual diastereomers. All such isomeric forms of these compounds are expressly included in the present invention. Each stereogenic carbon may be of the R or S configuration. The explicitly shown hydroxyl is also preferred to be syn to D, in the extended zig-zag conformation between the nitrogens shown in compounds of formula I.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a mammal or for use in affinity chromatography applications). Typically, such compounds are stable at a temperature of 40° C. or less, in the absence of moisture or other chemically reactive conditions, for at least a week.

The compounds of the present invention may be used in the form of salts derived from inorganic or organic acids. Included among such acid salts, for example, are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate.

This invention also envisions the quaternization of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chlorides, bromides and iodides; dialkyl sulfates including dimethyl, diethyl, dibutyl and diamyl sulfates; long chain halides such as decyl, lauryl, myrist_ and stearyl chlorides, bromides and iodides; and aralkyl halides including benzyl and phenethyl bromides. Water or oil-soluble or dispersible products may be obtained by such quaternization.

The novel sulfonamides of this invention are those of formula I:

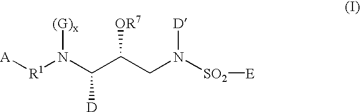

wherein:

each R¹ is independently selected from the group consisting of C(O)—, —S(O)₂—, —C(O)—C(O)—, —O—C(O)—, —O—S(O)₂, —NR²—S(O)₂—, —NR²—C(O)— and —NR²—C(O)—C(O)—;

each A is independently selected from the group consisting of 5-7 membered monocyclic heterocycles containing from 1-3 endocyclic heteroatoms, which may be optionally methylated at the point of attachment, optionally benzofused, optionally attached through a C₁-C₃ alkyl linker and optionally fused with a 5-7 membered monocyclic heterocycle containing from 1-2 endocyclic heteroatoms, and wherein unmethylated THF is expressly excluded;

each Ht is independently selected from C₃-C₇ cycloalkyl; C₅-C₇ cycloalkenyl; C₆-C₁₀ aryl; or a 5-7 membered saturated or unsaturated heterocycle, containing one or more heteroatoms selected from N, N(R²), O, S and S(O)ₙ; wherein said aryl or said heterocycle is optionally fused to Q; and wherein any member of said Ht is optionally substituted with one or more substituents independently selected from oxo, —OR², SR², —R², —N(R²)(R²), —R²—OH, —CN, —CO₂R², —C(O)—N(R²)₂, —S(O)₂—N(R²)₂, —N(R²)—C(O)—R², —C(O)—R², —S(O)ₙ—R², —OCF₃, —S(O)ₙ—Q, methylenedioxy, —N(R²)—S(O)₂(R²), halo, —CF₃, —NO₂, Q, —OQ, —OR⁷, —SR⁷, —R⁷, —N(R²)(R⁷) or —N(R⁷)₂;

each Q is independently selected from a 3-7 membered saturated, partially saturated or unsaturated carbocyclic ring system; or a 5-7 membered saturated, partially saturated or unsaturated heterocyclic ring containing one or more heteroatoms selected from O, N, S, S(O)ₙ or N(R²); wherein Q is optionally substituted with one or more groups selected from oxo, —OR², —R², —N(R²)₂, —N(R²)—C(O)—R², —R²—OH, —CN, —CO₂R², —C(O)—N(R²)₂, halo or —CF₃;

each R² is independently selected from the group consisting of H and C₁-C₃ alkyl optionally substituted with Q;

each x is independently 0 or 1;

each R³ is independently selected from the group consisting of H, Ht, C₁-C₆ alkyl and C₂-C₆ alkenyl wherein any member of said R³, except H, may be optionally substituted with one or more substituents selected from the group consisting of —OR², —C(O)—NH—R², —S(O)ₙ—N(R²)(R²), Ht, —CN, —SR², —CO₂R², NR²—C(O)—R²;

each n is independently 1 or 2;

G, when present, is selected from H, R⁷ or C₁-C₄ alkyl, or, when G is C₁-C₄ alkyl, G and R⁷ are bound to one another either directly or through a C₁-C₃ linker to form a heterocyclic ring; or

when G is not present (i.e., when x in (G)ₓ is 0), then the nitrogen to which G is attached is bound directly to the R⁷ group on —OR⁷;

each D and D′ is independently selected from the group consisting of Q; C₁-C₅ alkyl, which may be optionally substituted with one or more groups selected from C₃-C₆ cycloalkyl, —OR², —R³, —O—Q, —S—Q and Q; C₂-C₄ alkenyl, which may be optionally substituted with one or more groups selected from the group consisting of C₃-C₆ cycloalkyl, —OR², R³, O—Q and Q; C₃-C₆ cycloalkyl, which may be optionally substituted with or fused with Q; and C₅-C₆ cycloalkenyl, which may be optionally substituted with or fused with R⁶;

each E is independently selected from the group consisting of Ht; —O—Ht; Ht—Ht; —O—R³; —NR²R³; C₁-C₆ alkyl, which may be optionally substituted with one or more groups selected from the group consisting of R⁴ and Ht; and C₂-C₆ alkenyl, which may be optionally substituted with one or more groups selected from the group consisting of R⁴ and Ht; C₃-C₆ saturated carbocycle, which is optionally substituted with one or more groups selected from R⁴ or Ht; or C₅-C₆ unsaturated carbocycle, which is optionally substituted with one or more groups selected from R⁴ or Ht;

each R⁴ is independently selected from the group consisting of OR², —C(O)—NHR², S(O)₂—NHR², halo, NR²—C(O)—R² and —CN;

each R⁵ is independently selected from the group consisting of H and C₁-C₄ alkyl optionally substituted with aryl; and

each R⁶ is independently selected from the group consisting of aryl, carbocycle and heterocycle, wherein said aryl, carbocycle or heterocycle may be optionally substituted with one or more groups selected from the group consisting of oxo, —OR⁵, —R⁵, N(R⁵)(R⁵), N(R⁵)—C(O)—R⁵, —R⁵—OH, —CN, CO₂R⁵, C(O)—N(R⁵)(R⁵), halo and CF₃;

each R is independently selected from

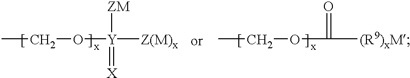

wherein each M is independently selected from H, Li, Na, K, Mg, Ca, Ba, —N(R²)₄, C₁-C₁₂-alkyl, C₂-C₁₂-alkenyl, —R⁶; wherein 1 to 4 —CH₂ radicals of the alkyl or alkenyl group, other than the —CH₂ that is bound to Z, is optionally replaced by a heteroatom group selected from O, S, S(O), S(O₂), or N(R²); and wherein any hydrogen in said alkyl, alkenyl or R⁶ is optionally replaced with a substituent selected from oxo, —OR², —R², N(R²)₂, N(R²)₃, R²OH, —CN, —CO₂R², —C(O)—N(R²)₂, S(O)₂—N(R²)₂, N(R²)—C(O)—R₂, C(O)R², —S(O)ₙ—R², OCF₃, —S(O)ₙ—R⁶, N(R²)—S(O)₂(R²), halo, —CF₃, or —NO₂;

M′ is H, C₁-C₁₂-alkyl, C₂-C₁₂-alkenyl, —R⁶; wherein 1 to 4 —CH₂ radicals of the alkyl or alkenyl group is optionally replaced by a heteroatom group selected from O, S, S(O), S(O₂), or N(R²); and wherein any hydrogen in said alkyl, alkenyl or R⁶ is optionally replaced with a substituent selected from oxo, —OR², —R², —N(R²)₂, N(R²)₃, —R²OH, —CN, —CO₂R², —C(O)—N(R²)₂, —S(O)₂—N(R²)₂, —N(R²)—C(O)—R₂, —C(O)R², —S(O)ₙ—R², —OCF₃, —S(O)ₙ—R⁶, —N(R²)—S(O)₂(R²), halo, —CF₃, or —NO₂;

Z is O, S, N(R²)₂, or, when M is absent, H;

Y is P or S;

X is O or S; and

R⁹ is C(R²)₂, O or N(R²); and wherein when Y is S, Z is not S; and

R⁶ is a 5-6 membered saturated, partially saturated or unsaturated carbocyclic or heterocyclic ring system, or an 8-10 membered saturated, partially saturated or unsaturated bicyclic ring system; wherein any of said heterocyclic ring systems contains one or more heteroatoms selected from O, N, S, S(O)ₙ or N(R²); and wherein any of said ring systems optionally contains 1 to 4 substituents independently selected from OH, C₁-C₄ alkyl, O—C₁-C₄ alkyl or OC(O)C₁-C₄ alkyl.

Preferred compounds of formula I have the following definitions for one or more of the below-specified substituents:

each R¹ is —O—C—(O)—;

each A is independently selected from the group consisting of 5-6 membered monocyclic heterocycles containing from 1-2 endocyclic oxygen atoms, which may be optionally methylated at the point of attachment, optionally attached through a C₁-C₃ alkyl linker and optionally fused with a 5-6 membered monocyclic heterocycle containing from 1-2 endocyclic oxygen atoms, and more preferably, A is selected from the group consisting of dioxanyl (preferably, 1,3-dioxanyl), dioxolanyl, dioxolanylmethyl, 3-methyl THF, tetrahydrofurofuranyl, tetrahydrofurodihydrofuranyl, tetrahydropyranofuranyl, tetrahydropyranodihydrofuranyl, pyranyl, dihydropyranyl and tetrahydropyranyl. Most preferably, A is 1,3-dioxanyl attached at the 5-position.

each D is C₁-C₅ alkyl, which may be optionally substituted with one or more Ht, more preferably D is C₁-C₅ alkyl, which may be optionally substituted with one group selected from C₆-C₁₀ aryl and C₃-C₆ cycloalkyl, even more preferably D is selected from benzyl, isobutyl, cyclopentylmethyl, and cyclohexylmethyl and most preferably, D is benzyl or isobutyl;

each D′ is selected from the group consisting of C₁-C₆ alkyl optionally substituted with R⁶ (wherein each R⁶ is independently selected from the group consisting of carbocycle and heterocycle, wherein said heterocycle or carbocycle may be optionally substituted with one or more groups selected from the group consisting of oxo, OR⁵, —R⁵, N(R⁵)(R⁵), N(R⁵)—C(O)—R⁵, —R⁵—OH, —CN, CO₂R⁵, C(O)—N(R⁵)(R⁵), halo and CF₃ and each R⁵ is independently selected from the group consisting of H and C₁-C₃ alkyl), and more preferably D′ is selected from the group consisting of C₁C₄ alkyl optionally substituted with one 3-6 membered carbocycle or one 5-6 membered heterocycle, and most preferably, D′ is selected from the group consisting of isobutyl, cyclopentylmethyl and cyclohexylmethyl;

each E is Ht and more preferably, E is phenyl substituted with 0-2 substituents chosen from the group consisting of OH, OR⁷ OCH₃, NH₂, NHCOCH₃, SCH₃, and CH₃; or phenyl fused with 5-6 membered heterocycle, and even more preferably, E is phenyl substituted with one substituent selected from the group consisting of OH, OR⁷ OCH₃, NH₂, NHCOCH₃, SCH₃, and CH₃; or phenyl fused with 5-6 membered heterocycle, and most preferably, E is phenyl substituted with NH₂, NHR⁷ or N(R⁷)₂ (preferably in the meta- or para-position).

Preferably R⁷ is

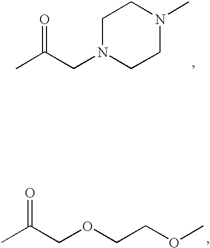

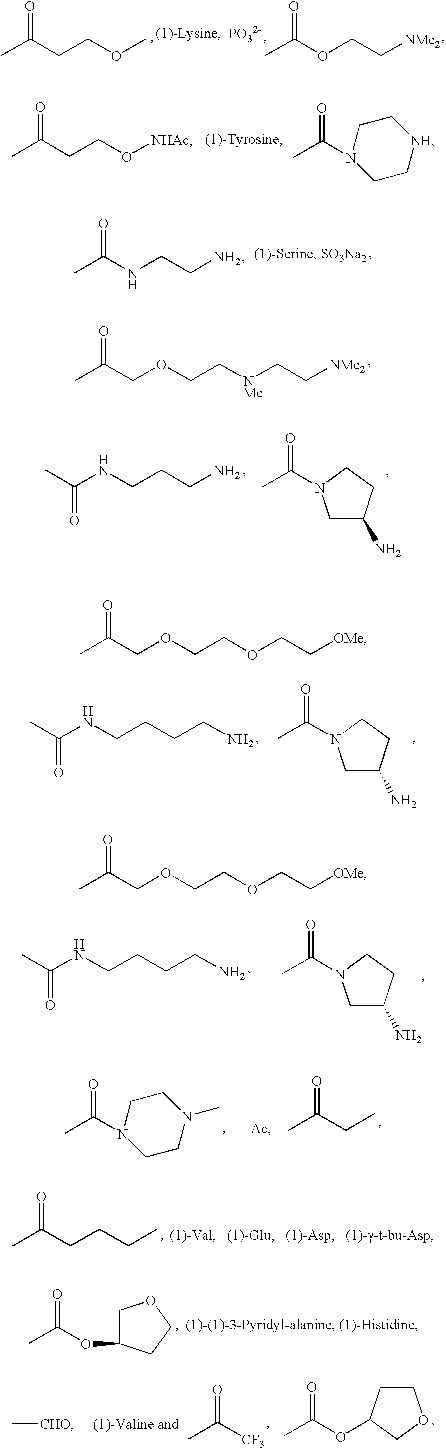

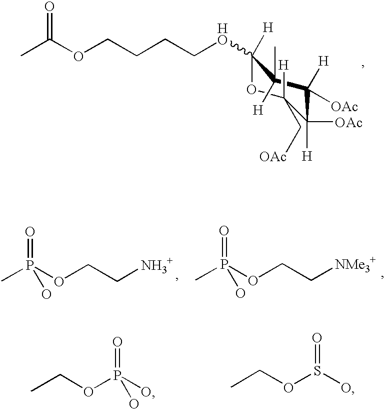

PO₃K₂, PO₃Ca, PO₃-spermine, P₃-(spermidine)₂ or PO₃-(meglamine)₂.

It will be understood by those of skill in the art that component M or M′ in the formulae set forth herein will have either a covalent, a covalent/zwitterionic, or an ionic association with either Z or R⁹ depending upon the actual choice for M or M′. When M or M′ is hydrogen, alkyl, alkenyl, or R⁶, M or M′ is covalently bound to R⁹ or Z. If M is a mono- or bivalent metal or other charged species (i.e., NH₄⁺), there is an ionic interaction between M and Z and the resulting compound is a salt.

When x is 0 in (M)ₓ, Z may be a charged species. When that occurs, the other M may be oppositely charged to produce a 0 net charge on the molecule. Alternatively, the counter ion may located elsewhere in the molecule.

Except where expressly provided to the contrary, as used herein, the definitions of variables A, R¹-R⁴, R⁶-R⁹, Ht, B, x, n, D, D′, M, Q, X, Y, Z and E are to be taken as they are defined above for the compounds of formula I.

Table I illustrates preferred compounds of this invention:

TABLE 1

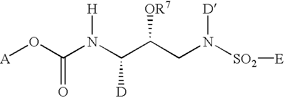

| COMPOUND | A | D | D′ | E |
|---|---|---|---|---|
| 1 | 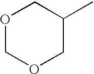 | 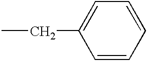 | 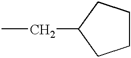 | 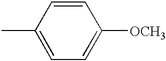 |
| 2 | 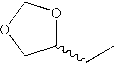 | 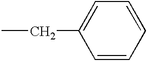 | 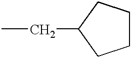 | 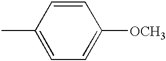 |
| 3 | 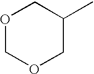 | 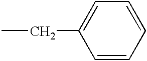 | 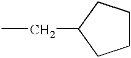 | 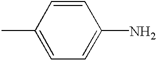 |
| 4 | 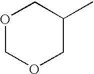 | 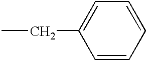 | 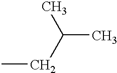 | 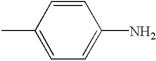 |
| 5 | 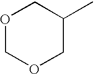 | 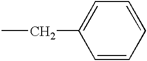 | 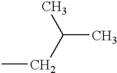 | 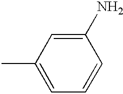 |
| 6 | 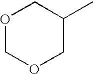 | 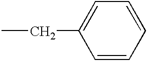 | 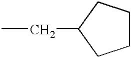 | 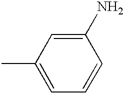 |
| 7 (Isomer A) | 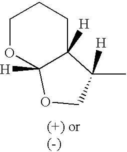 | 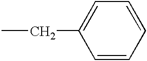 | 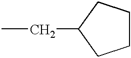 | 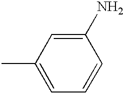 |
| 8 (Isomer B) | 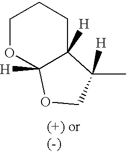 | 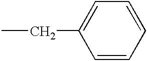 | 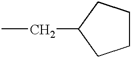 | 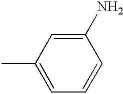 |
| 9 (Isomer A) | 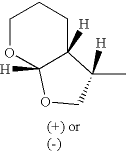 | 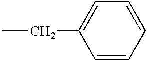 | 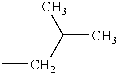 | 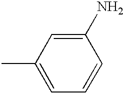 |
| 10 (Isomer B) | 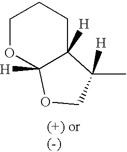 | 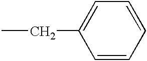 | 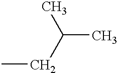 | 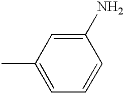 |
| 11 | 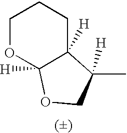 | 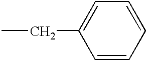 | —CH₃ | 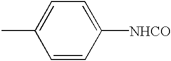 |
| 12 | 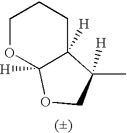 | 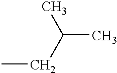 | 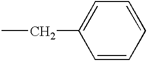 | 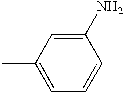 |
| 13 | 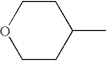 | 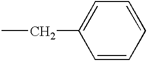 | 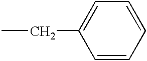 | 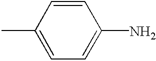 |
| 14 | 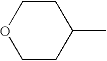 | 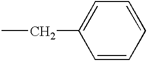 | 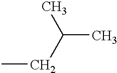 | 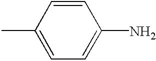 |
| 15 | 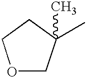 | 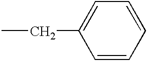 | 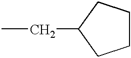 | 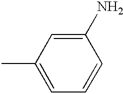 |
| 16 | 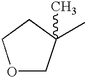 | 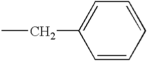 | 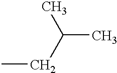 | 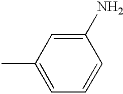 |

The prodrugs of the present invention may be synthesized using conventional synthetic techniques. WO 96/33187 discloses the synthesis of compounds of formula:

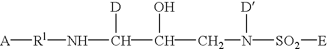

wherein A, R¹, D, D′ and E are as defined above. Prodrugs of formula (I) of the present invention can be readily synthesized from the '187 compounds using conventional techniques. One of skill in the art would be well aware of conventional synthetic reagents to convert the —OH group of the '187 compounds to a desired —OR⁷ functionality of the present invention, wherein R⁷ is as defined above. The relative ease with which the compounds of this invention can be synthesized represents an enormous advantage in the large scale production of these compounds.

For example, VX-478, a compound disclosed in U.S. Pat. No. 5,585,397, can be readily converted to the corresponding bis-phosphate ester derivative, as shown below:

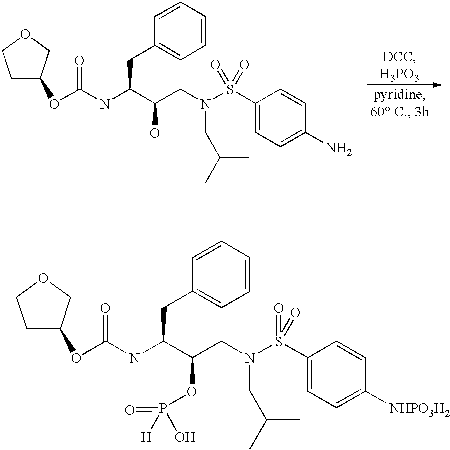

Alternatively, if the monophosphate ester of VX-478 is desired, then the synthetic scheme can be readily adapted by beginning with the 4-nitrophenyl derivative of VX-478, as shown below:

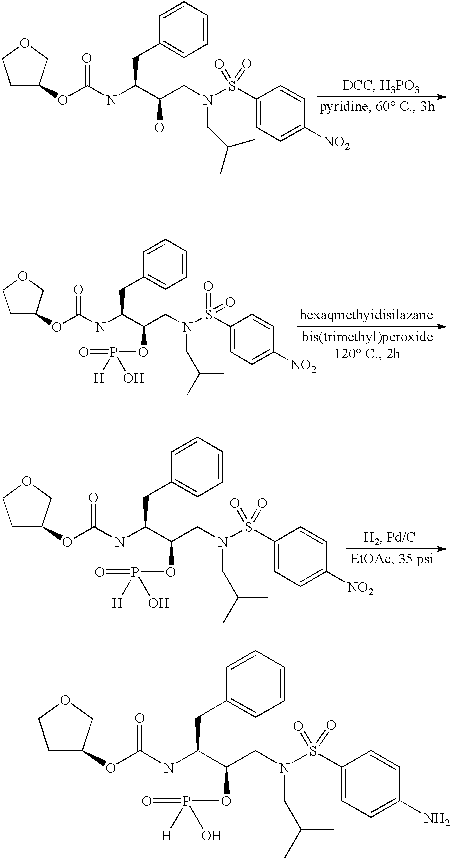

Although unmethylated tetrahydrofuran embodiments of formula I, such as VX 478, are expressly excluded from the present invention, one of skill in the art would readily be able to prepare the corresponding monophosphate and bis-phosphate esters of the present invention using similar reaction conditions.

Further examples of specific compounds which may be converted to the prodrugs of this invention by similar techniques (and the syntheses of those intermediates to the compounds of the present invention) are disclosed in WO 94/05639 and '397 patent, the disclosures of which are herein incorporated by reference.

Pharmaceutically acceptable salts of the compounds of the present invention may be readily prepared using known techniques. For example, the disodium salt of the mono-phosphate ester shown above can be prepared as shown below:

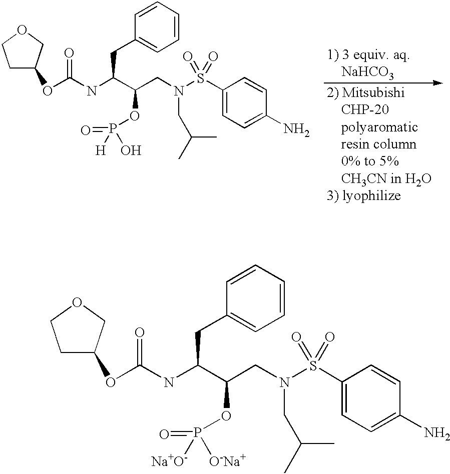

The compounds of this invention may be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

Without being bound by theory, we believe that two different mechanisms are involved in converting the prodrugs of this invention into the active drug, depending upon the structure of the prodrug. The first mechanism involves the enzymatic or chemical transformation of the prodrug species into the active form. The second mechanism involves the enzymatic or chemical cleavage of a functionality on the prodrug to produce the active compound.

The chemical or enzymatic transformation can involve to transfer of a functional group (i.e., R⁷) from one heteroatom within the molecule to another heteroatom. This transfer is demonstrated in the chemical reactions shown below:

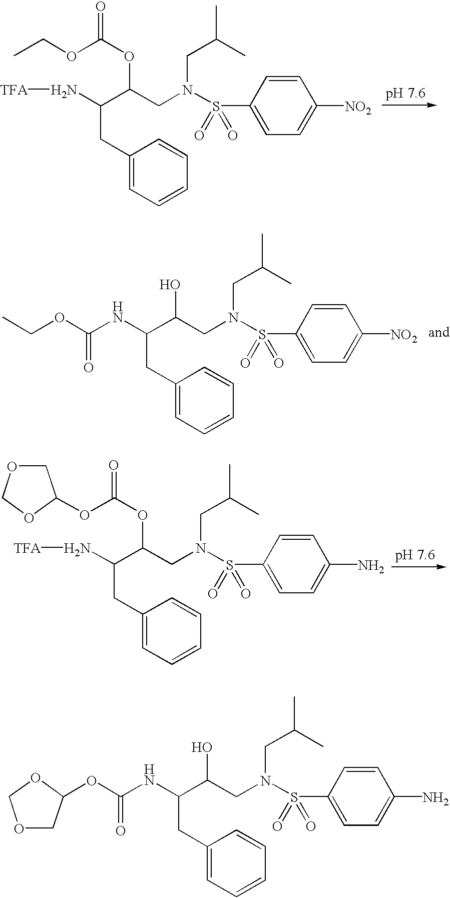

The cleavage mechanism is demonstrated by the reaction below where a phosphate ester-containing prodrug is converted into the active form of the drug by removal of the phosphate group.

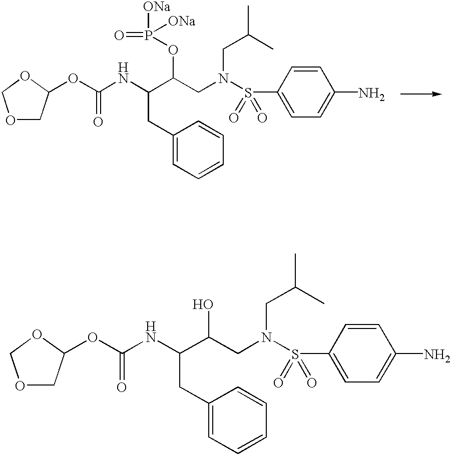

These protease inhibitors and their utility as inhibitors of aspartyl proteases are described in WO 96/33187, the disclosure of which is incorporated herein by reference.

The prodrugs of the present invention are characterized by unexpectedly high aqueous solubility. This solubility facilitates administration of higher doses of the prodrug, resulting in a greater drug load per unit dosage. The prodrugs of the present invention are also characterized by facile hydrolytic cleavage to release the active aspartyl protease inhibitor in vivo. The high aqueous solubility and the facile in vivo metabolism result in a greater bioavailability of the drug. As a result, the pill burden on a patient is significantly reduced.

The prodrugs of this invention may be employed in a conventional manner for the treatment of viruses, such as HIV and HTLV, which depend on aspartyl proteases for obligatory events in their life cycle. Such methods of treatment, their dosage levels and requirements may be selected by those of ordinary skill in the art from available methods and techniques. For example, a prodrug of this invention may be combined with a pharmaceutically acceptable adjuvant for administration to a virally-infected patient in a pharmaceutically acceptable manner and in an amount effective to lessen the severity of the viral infection.

Alternatively, the prodrugs of this invention may be used in vaccines and methods for protecting individuals against viral infection over an extended period of time. The prodrugs may be employed in such vaccines either alone or together with other compounds of this invention in a manner consistent with the conventional utilization of protease inhibitors in vaccines. For example, a prodrug of this invention may be combined with pharmaceutically acceptable adjuvants conventionally employed in vaccines and administered in prophylactically effective amounts to protect individuals over an extended period time against HIV infection. As such, the novel protease inhibitors of this invention can be administered as agents for treating or preventing HIV infection in a mammal.

The prodrugs of this invention may be administered to a healthy or HIV-infected patient either as a single agent or in combination with other anti-viral agents which interfere with the replication cycle of HIV. By administering the compounds of this invention with other anti-viral agents which target different events in the viral life cycle, the therapeutic effect of these compounds is potentiated. For instance, the co-administered anti-viral agent can be one which targets early events in the life cycle of the virus, such as cell entry, reverse transcription and viral DNA integration into cellular DNA. Anti-HIV agents targeting such early life cycle events include, didanosine (ddI), alcitabine (ddC), d4T, zidovudine (AZT), polysulfated polysaccharides, sT4 (soluble CD4), ganiclovir, dideoxycytidine, trisodium phosphonoformate, eflornithine, ribavirin, acyclovir, alpha interferon and trimenotrexate. Additionally, non-nucleoside inhibitors of reverse transcriptase, such as TIBO or nevirapine, may be used to potentiate the effect of the compounds of this invention, as may viral uncoating inhibitors, inhibitors of trans-activating proteins such as tat or rev, or inhibitors of the viral integrase.

Combination therapies according to this invention exert a synergistic effect in inhibiting HIV replication because each component agent of the combination acts on a different site of HIV replication. The use of such combinations also advantageously reduces the dosage of a given conventional anti-retroviral agent which would be required for a desired therapeutic or prophylactic effect as compared to when that agent is administered as a monotherapy. These combinations may reduce or eliminate the side effects of conventional single anti-retroviral agent therapies while not interfering with the anti-retroviral activity of those agents. These combinations reduce potential of resistance to single agent therapies, while minimizing any associated toxicity. These combinations may also increase the efficacy of the conventional agent without increasing the associated toxicity. In particular, we have discovered that these prodrugs act synergistically in preventing the replication of HIV in human T cells. Preferred combination therapies include the administration of a prodrug of this invention with AZT, ddI, ddC or d4T.

Alternatively, the prodrugs of this invention may also be co-administered with other HIV protease inhibitors such as Ro 31-8959 (Roche), L-735,524 (Merck), XM 323 (Du-Pont Merck) and A-80,987 (Abbott) to increase the effect of therapy or prophylaxis against various viral mutants or members of other HIV quasi species.

We prefer administering the prodrugs of this invention as single agents or in combination with retroviral reverse transcriptase inhibitors, such as derivatives of AZT, or other HIV aspartyl protease inhibitors. We believe that the co-administration of the compounds of this invention with retroviral reverse transcriptase inhibitors or HIV aspartyl protease inhibitors may exert a substantial synergistic effect, thereby preventing, substantially reducing, or completely eliminating viral infectivity and its associated symptoms.

The prodrugs of this invention can also be administered in combination with immunomodulators (e.g., bropirimine, anti-human alpha interferon antibody, IL-2, GM-CSF, methionine enkephalin, interferon alpha, diethyldithiocarbamate, tumor necrosis factor, naltrexone and rEPO); and antibiotics (e.g., pentamidine isethiorate) to prevent or combat infection and disease associated with HIV infections, such as AIDS and ARC.

When the prodrugs of this invention are administered in combination therapies with other agents, they may be administered sequentially or concurrently to the patient. Alternatively, pharmaceutical or prophylactic compositions according to this invention may be comprised of a combination of a prodrug of this invention and another therapeutic or prophylactic agent.

Although this invention focuses on the use of the prodrugs disclosed herein for preventing and treating HIV infection, the compounds of this invention can also be used as inhibitory agents for other viruses which depend on similar aspartyl proteases for obligatory events in their life cycle. These viruses include, as well as other AIDS-like diseases caused by retroviruses, such as simian immunodeficiency viruses, but are not limited to, HTLV-I and HTLV-II. In addition, the compounds of this invention may also be used to inhibit other aspartyl proteases, and in particular, other human aspartyl proteases, including renin and aspartyl proteases that process endothelin precursors.

Pharmaceutical compositions of this invention comprise any of the compounds of the present invention, and pharmaceutically acceptable salts thereof, with any pharmaceutically acceptable carrier, adjuvant or vehicle. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered orally, parenterally, by inhalation spray, topically, rectally, nasally, buccally, vaginally or via an implanted reservoir. We prefer oral administration or administration by injection. The pharmaceutical compositions of this invention may contain any conventional non-toxic pharmaceutically-acceptable carriers, adjuvants or vehicles. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Dosage levels of between about 0.01 and about 100 mg/kg body weight per day, preferably between about 0.5 and about 50 mg/kg body weight per day of the active ingredient compound are useful in the prevention and treatment of viral infection, including HIV infection. Typically, the pharmaceutical compositions of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w), Preferably, such preparations contain from about 20% to about 80% active compound.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

As the skilled artisan will appreciate, lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the infection, the patient's disposition to the infection and the judgment of the treating physician.

In order that this invention be more fully understood, the following examples are set forth. These examples are for the purpose of illustration only and are not to be construed as limiting the scope of the invention in any way.

### EXAMPLE 1

General Conditions:

(A) Analytical HPLC 0-100%B/30 min, 1.5 mL/min, A=0.1% TFA in water, B=0.1% TFA in acetonitrile. Detection at 254 and 220 nm, C18 reverse phase Vydac, t0=2.4 min.

(B) ⅓ v/v EtOAc/hexane

(C) ½ v/v EtOAc/hexane

(D) Analytical HPLC 0-100%B/10 min, 1.5 mL/min, A=0.1% TFA in water, B=0.1% TFA in acetonitrile. Detection at 254 and 220 nm, C18 reverse phase Vydac, t₀=2.4 min.

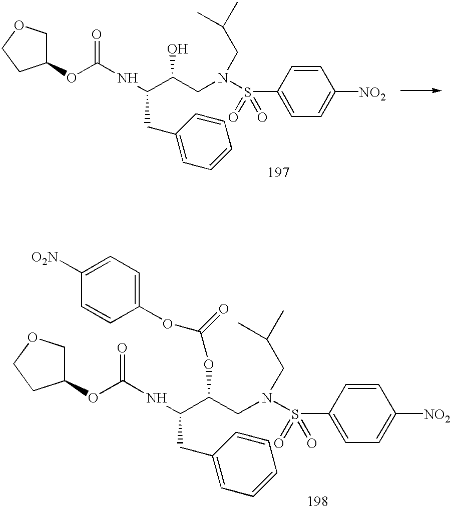

A mixture of 2.0 g (3.7 mMol) of 197 and 3.0 g (16 mMol) of di-p-nitrophenyl carbonate in 10 ml of dimethylformamide was treated at 25° with 4 ml (4 mMol) of P4-phosphazene base (Fluka, 1M in hexane). The mixture was stirred for 6 h at 25° until all of the starting alcohol was consumed. The reaction mixture was partitioned between ethyl acetate and 1N hydrochloric acid. The organic layer was washed with 1N sodium hydroxide and brine, dried over magnesium sulfate and concentrated in vacuo. Titration with dichloromethane gave the desired mixed carbonate (1.2 g crop1 and 0.6 g crop 2) as a fine powder. Combined yield: 69%. Rf=0.13 (⅓ EtOAc/hexane, conditions B), Rf=0.40 (½ EtOAc/hexane, conditions C), tHPLC=23.83 min (A), MS(ES+) 701 (M+1).

1H-NMR (CDCl3): 0.82 (6H,dd), 1.9 (2H,m), 2.15 (1H,m), 2.8 (1H,m), 3.0 (4H,m), 3.5 (2H,m), 3.6 (1H,m), 3.8 (4H,m), 4.3 (1H,bs), 4.8 (1H,m), 5.17 (2H,m), 7.7 (7H,m), 7.95 (2H,d), 8.35 (4H,m).

13C (CDCl3): 155.2 152.2, 149.9, 145.6, 135.9, +129.0, +128.8, +128.5, +127.2, +125.4, +124.4, +121.8, +78.1, +75.8, −73.1, −66.9, −56.5, +52.7, −48.2, −35.9, −35.9, 32.6, −+26.4, +19.9, +19.8.

### EXAMPLE 2

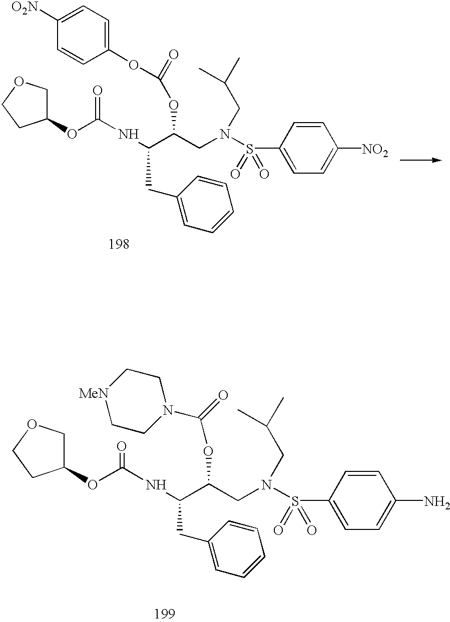

To 0.20 g (0.286 mM) of 198 dissolved in 3 ml of THF was added 0.11 g (1.14 mM) of 1-Methyl-piperidine and the mixture was stirred overnight at room temperature (“rt”). All the solvents were then evaporated and the solid residue partitioned between EtOAc and water. The volatiles were removed and, where appropriate, the residue was treated with 1:1 TFA/DCM over 30 min at rt to remove the Boc protecting group. The product was dissolved in 0.25 ml TFA and 1.5 ml THF. Hydrogenolysis for 10 hours in presence of 30 mg of 10% Pd/C gave the desired compound. The final purification was on preparative reversed phase C18 using conditions Example 1, except that the flow rate was 18 ml/min.

C,H,N: calc: 49.27, 5.57, 8.25, found 49.15, 5.76, 8.29 C₃₁H₄₅N₅O₇S₁. 1.9CF₃COOH

LC/MS (ES+) 632 (M+1) 1 peak at 4.71 min

Analytical HPLC(A) t=N/A min

1H:0.71 (3H,d), 0.74 (3H,d), 1.80 (2H,m), 2.03 (1H,m), 2.63 (2H,m), 2.74 (1H,m), 2.82 (3H,s), 2.92 (2H,m), 3.20 (4H,m), 3.42 (3H,m), 3.62 (2H,m), 3.75 (1H,m), 4.05 (3H,m), 4.97 (2H,m), 6.2 (1H,bs), 6.60 (2H,m), 7.22 (5H,m), 7.40 (3H,m), 13C (DMSO): 156.4, 154.0, 153.8, 138.8, 129.6, 129.5, 128.3, 126.5, 123.7, 112.7, 74.8, 72.9, 66.7, 58.2, 54.0, 53.1, 49.3, 42.3, 40.8, 36.0, 33.3, 25.8, 20.4, 20.3

### EXAMPLE 3

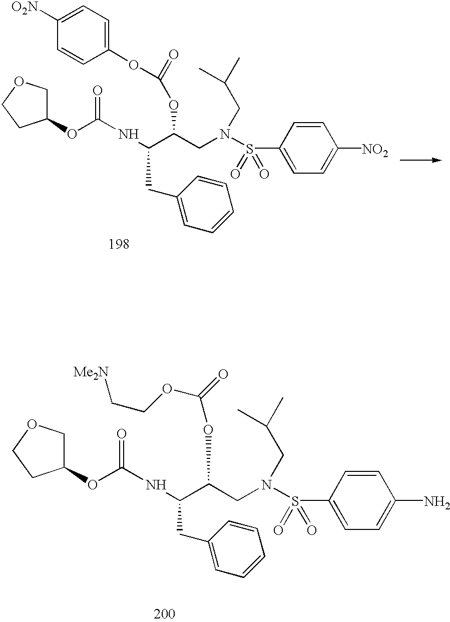

The synthesis of compound 200 from compound 198 was carried as described in Example 1, except that N,N-dimethyl-aminoethanol was used in place of di-p-nitrophenyl carbonate.

1HNMR (acetone-d6): 0.82 (6H,dd), 1.83 (2H,m), 2.07 (1H,m), 2.64 (2H,m), 2.82 (6H,s), 2.90 (2H,m), 3.19 (1H,m), 3.38 (4H,m), 3.63 (2H,m), 3.76 (1H,m), 4.17 (2YH,m), 4.40 (1H,m), 4.56 (1H,m), 4.96 (1H,m), 5.06 (1H,m), 6.06 (1H,d), 6.68 (2H,d), 7.23 (5H,m), 7.47 (2H,d).

13CNMR (acetone d6): 20.2, 20.3, 27.5, 33.4, 35.6, 43.8, 50.1, 54.2, 56.4, 58.5, 63.1, 67.4, 73.6, 76.2, 79.9, 114.2, 118.3, 127.4, 129,2, 130.1, 130.3, 139.3, 153.4, 157.0.

LC/MS: 1 peak, 621 (MH+).

### EXAMPLE 4

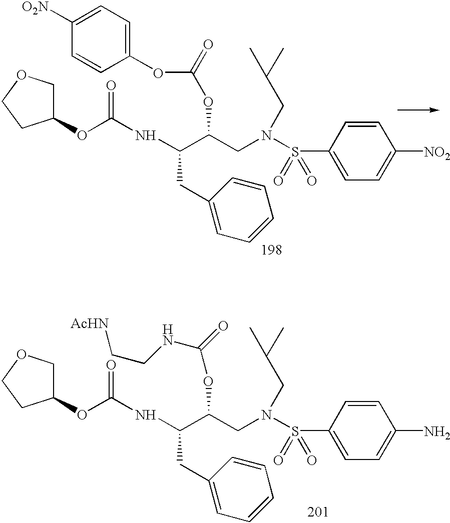

The synthesis of compound 201 from compound 198 was carried as described in Example 1, except that N-acetyl-ethylenediamine was used in place of di-p-nitrophenyl carbonate,

C,H,N: calc: 49.66, 5.64, 8.83, found 49.76, 5.98, 8.93 C₃₀H₄₃N₅O₈S₁. 1.4CF₃COOH.

LC/MS (ES+) 634 (M+1) 1 peak at 5.08 min.

Analytical HPLC(A)⁻ t=15.92 min.

1H: d-3 acetonitrile: 0.88 (6H,dd), 1.92 (3H,s), 1.94 (2H,m), 2.17 (1H,m), 2.72 (2H,m), 2.96 (2H,m), 3.07 (3H,m), 3.29 (1H,m), 3.42 (3H,m), 3.69 (1H,m), 3.77 (1H,m), 3.82 (1H,m), 4.133 (1H,m), 4.40 (1H,bs), 5.05 (2H,m), 5.80 (1H,m), 6.10 (1H,d), 6.78 (2H,d), 6.83 (1H,bs), 7.28 (5H,m), 7.58 (2H,d).

13C (d3-acetonitrile): 157.1, 157.0, 153.2, 139.6, +130.3, +130.2, +129.2, +127.2, 126.2, +114.2, +76.0, +75.4, −73.6, −67.4, −58.2, +54.9, −50.2, −41.6, −39.8, −35.9, −33.4, +27.3, +23.1, +20.4, +20.2.

### EXAMPLE 5

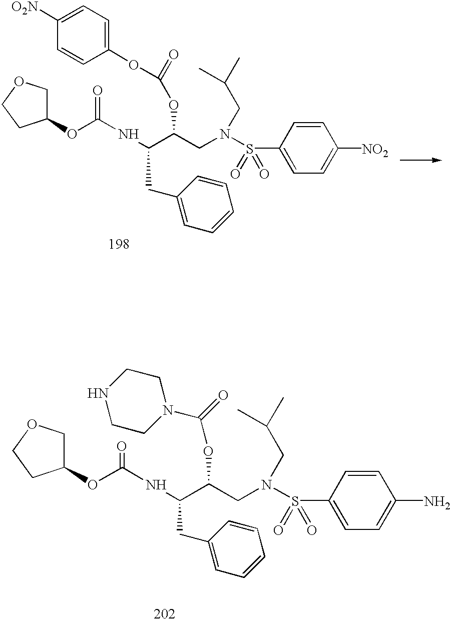

The synthesis of compound 202 from compound 198 was carried as described in Example 1, except that mono N-Boc-piperazine was used in place of di-p-nitrophenyl carbonate.

C,H,N: calc: 48.28, 5.68, 8.41, found 48.28, 5.36, 8.28 C₃₀H₄₃N₅O₇S₁×2 CF₃COOH

LC/MS (ES+) 618 (M+1) 1 peak at 4.36 min.

Analytical HPLC(A) t=14.84 min.

1H: d6-DMSO: 0.72 (3H,d), 0.77 (3H,d), 1.78 (2H,m), 2.09 (1H,m), 2.64 (2H,m), 2.73 (1H,m), 2.80 (1H,m), 3.08 (4H,m), 3.32 (2H,m), 3.41 (1H,m), 3.50 (4H,m), 3.54 (1H,m), 3.63 (1H,m), 3.70 (1H,m), 3.98 (1H,m), 4.89 (1H,m), 4.97 (1H,m), 6.61 (2H,d), 7.23 (5H,m), 7.42 (3H,m), 8.88 (2H,bs).

13C: (DMSO): 155.7, 153.6, 153.0, 138.4, +129.1, +129.0, +128.1, +126.1, 123.2, +112.7, +75.2, +74.4, −72.5, −66.2, −56.9, +53.1, −48.8, −42.5, −40.8, −35.0, −32.2, +26.2, +20.0, +19.8.

### EXAMPLE 6

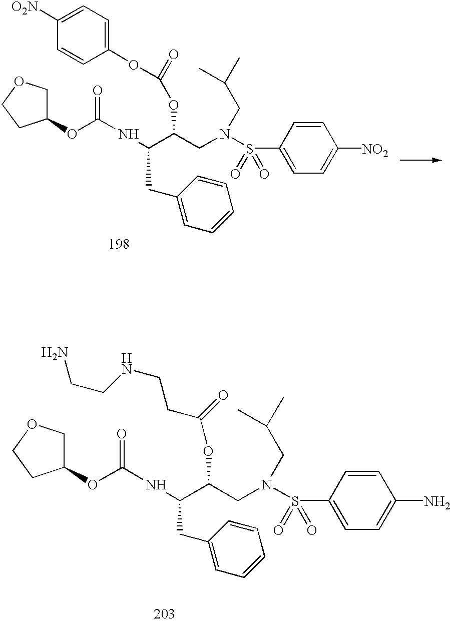

The synthesis of compound 203 from compound 198 was carried as described in Example 1, except that mono-N-Boc-ethylenediamine was used in place of di-p-nitrophenyl carbonate.

C,H,N: calc: 46.89, 5.29, 8.54, found 46.50, 5.51, 8.54 C₂₈H₄₁N₅O₇S₁×2 CF₃COOH.

LC/MS (ES+) 592 (M+1) 1 peak at 4.32 min.

Analytical HPLC(A) t=14.69 min.

1H:d-6 DMSO: 0.77 (6H,d), 1.82 (2H,m), 2.06 (1H,m), 2.57 (2H,m), 2.82 (4H,m), 2.97 (1H,m), 3.30 (5H,m), 3.55 (1H,m), 3.65 (1H,m), 3.70 (1H,m), 3.95 (1H,m), 4.88 (1H,m), 4.95 (1H,m), 6.62 (2H,d), 7.20 (6H,m), 7.39 (3H,m), 7.78 (3H,bs).

13C (dmso): 155.9, 152.9, 138.5, 129.2, 128.9, 128.1, 126.1, 122.9, 112.7, 74.7, 74.5, 72.6, 66.2, 57.2, 53.2, 49.4, 38.8, 37.94, 35.1, 32.1, 26.3, 20.0, 19.8.

### EXAMPLE 7

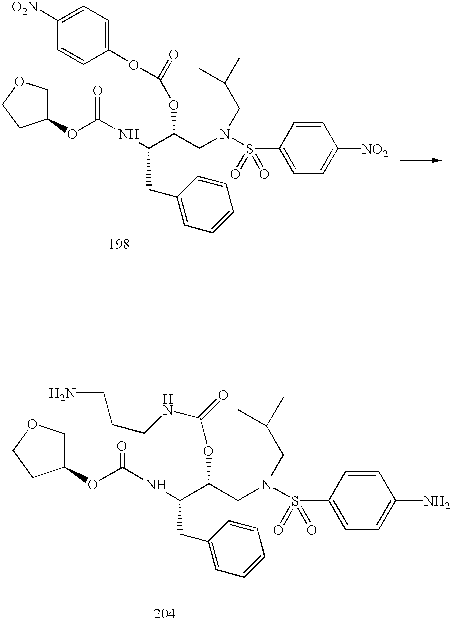

The synthesis of compound 204 from compound 198 was carried as described in Example 1, except that mono-1,3-diamino-3-N-Boc-propane was used in place of di-p-nitrophenyl carbonate.

C,H,N: calc: 49.07, 5.64, 8.89, found 48.95, 6.00, 8.92 C₂₉H₄₃N₅O₇S₁×1.6 CF₃COOH

LC/MS (ES+) 605 (M+1) 1 peak at 4.27 min.

Analytical HPLC(A) t=14.72 min.

1H:d-6 DMSO: 0.78 (6H,dd), 1.64 (2H,m), 1.83 (2H,m), 2.03 (1H,m), 2.57 (1H,m), 2,78 (4H,m), 2.94 (1H,m), 3.03 (2H,m), 3.32 (2H,m), 3.58 (1H,m), 3.63 (1H,m), 3.73 (1H,m), 3.87 (1H,m), 4.84 (1H,m), 4.92 (1H,m), 6.61 (2H,d), 7.22 (6H.m), 7.36 (1H,d), 7.28 (2H,d), 7.76 (3H,ns).

13C (dmso): 155.8, 155.7, 138.5, +129.1, +129.0, +128.0, +126.1, 122.9, +112.7, +74.6, +74.3, −72.7, −66.2, −57.2, +53.6, −49.5, −37.4, −36.7, −35.5, −32.1, −27.6, +26.2, +20.0, +19.8.

### EXAMPLE 8

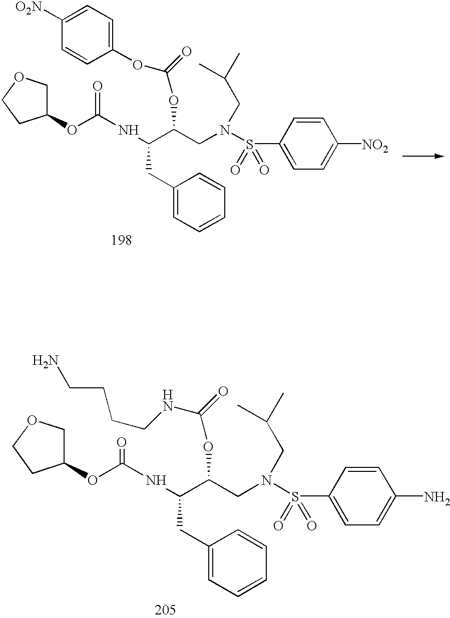

The synthesis of compound 205 from compound 198 was carried as described in Example 1, except that 1,4-diamino-4-N-Boc-butane was used in place of di-p-nitrophenyl carbonate.

C,H,N: calc: 48.17, 5.59, 8.26, found 48.02, 5.96, 8.24 C₃₀H₄₅N₅O₇S₁ 0.2 CF₃COOH

LC/MS (ES+) 620 (M+1) 1 peak at 4.36 min.

Analytical HPLC(A)⁻ t=14.93 min.

1H: d-6 DMSO: 0.77 (6H,dd), 1.43 (4H,m), 1.82 (2H,m), 2.03 (1H,m), 2.77 (4H,m), 2.95 (3H,m), 3.31 (2H,m), 3.56 (1H,m), 3.63 (1H,m), 3.70 (1H,bq), 3.82 (1H,m), 4.85 (1H,m), 4.92 (1H,m), 6.62 (2H,d), 7.2 (7H,m), 7.38 (2H,d), 7.72 (3H,bs).

13C: 155.7, 152.9, +138.6, +129.1, +129.0, +128.0, +126.1, +123.0, +112.7, +74.4, +74.3, −72.7, −66.2, −57.2, +53.7, −49.7, −38.6, −38.5, −35.4, −32.1, −26.3, +26.2, −24.4, +20.1, +19.9.

### EXAMPLE 9

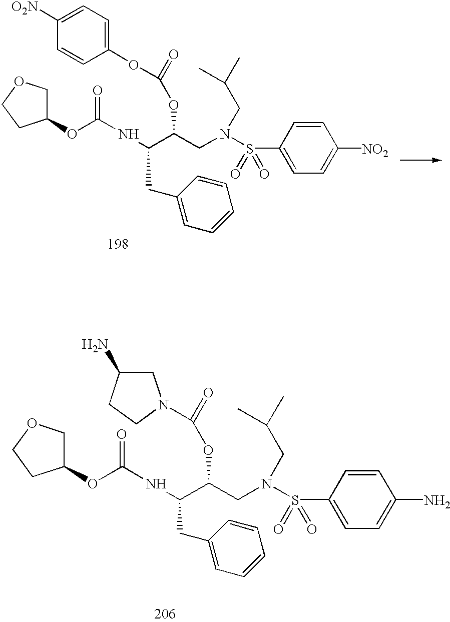

The synthesis of compound 206 from compound 198 was carried as described in Example 1, except that (3R)-(+)-3-Boc-aminopyrrolidine was used in place of di-p-nitrophenyl carbonate.

C,H,N: calc: 48.28, 5.36, 8.28, found 47.89, 5.53, 8.57 C₃₀H₄₃N₅O₇S₁×2 TFA

LC/MS (ES+) 618 (M+1) 1 peak at 4.32 min.

Analytical HPLC(A) t=14.31 min.

1H and 13C NMR: complex and overlapping mixtures of rotomers.

### EXAMPLE 10

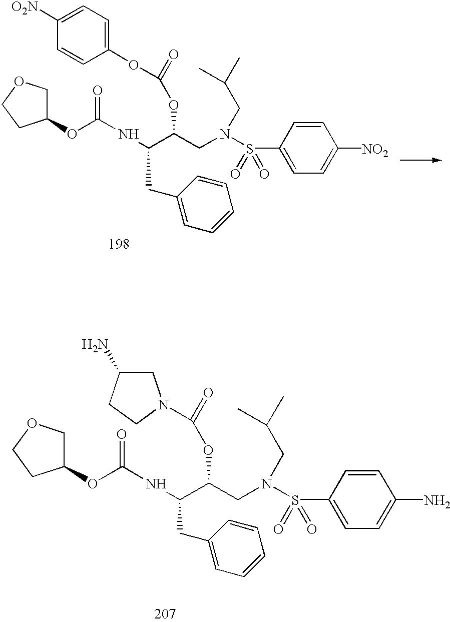

The synthesis of compound 207 from compound 198 was carried as described in Example 1, except that (3S)-(−)-3-Boc-aminopyrrolidine was used in place of di-p-nitrophenyl carbonate,

LC/MS (ES+) 618 (M+1) 1 peak at 4.19 min.

Analytical HPLC(A) t=14.75 min.

1H and 13C NMR: complex and overlapping mixtures of rotomers.

### EXAMPLE 11

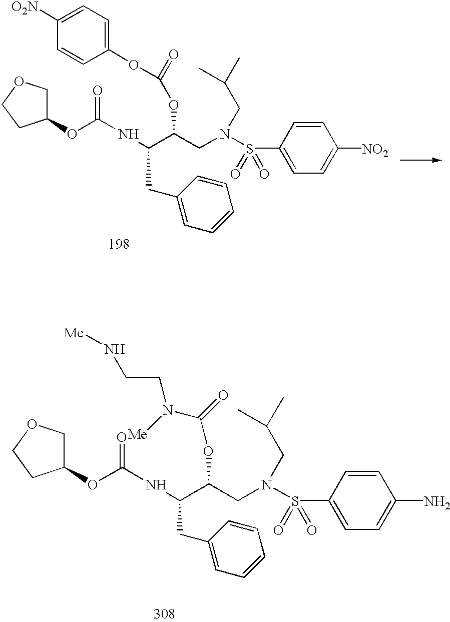

The synthesis of compound 308 from compound 198 was carried as described in Example 1, except that N-triphenylmethyl-N,N′-dimethylethanediamine was used in place of di-p-nitrophenyl carbonate.

1H-NMR: 0.76 (6H,dd), 1.65 (2H,m), 1.95 (1H,m), 2.07 (1H,m), 2.7 (2H,m), 2.75 (3H,s), 2.95 (3H,m), 3.45 (2H,m), 3.7 (4H,m), 4.2 (2H,bm), 5.05 (2H,bd), 6.62 (2H,d), 7.2 (5H,m)⁻, 7.5 (2H,d).

LC/MS: 1 peak, 620 (MH+).

### EXAMPLE 12

### General Procedures

Acylation:

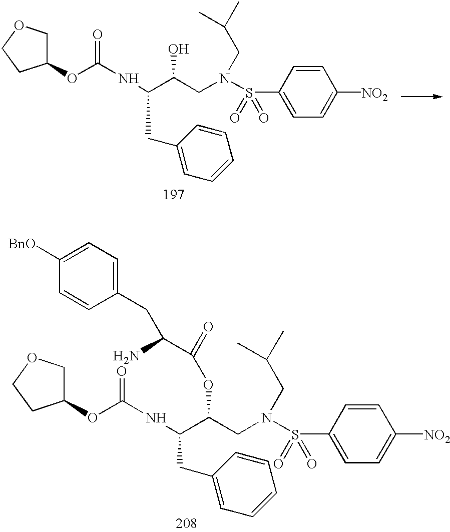

To 200 mg (0.37 mM) of 197 dissolved in 5 ml CH₂Cl₂ was added N-CBz-L-Benzyl tyrosine 183 mg (0.41 mM) followed by 231 mg (1.12 mM) DCC, followed by 29 mg (0.23 mM) DMAP. The reaction is stirred at rt for 24 hr. The precipitates present were removed by filtration. The filtrate was then concentrated in vacuo. The final compound was purified on preparative reversed phase C18 using purification by HPLC C18 Waters Delta Prep 3000 Column: YMC-Pack ODS AA 12S05-2520WT 250×20 mm I.D. S-5 mm, 120 Å, 0-100% B over ½ h, flow=18 ml/min, monitored at 220 nm, B=0.1 trifluoroacetic acid in acetonitrile, A=0.1% trifluoroacetic acid in water. Analytical Column: YMC-Pack ODS AA1 2S05-2520WT 250×4.6 mmI.D. S-5 mm, 120 Å, 0-100% B at 1.5 ml/min. over ½ h, monitored at 220 nm, B=0.1% trifluoroacetic acid in acetonitrile, A=0.1% trifluoroacetic acid in water.

The aqueous phase was lyophilized to give 59 mg, (16.3%) GW431896X, (U11484-72-10) tHPLC=11.71 min., MW=966.04, LC/MS=MH+967.

Reduction of the Nitro Functionality:

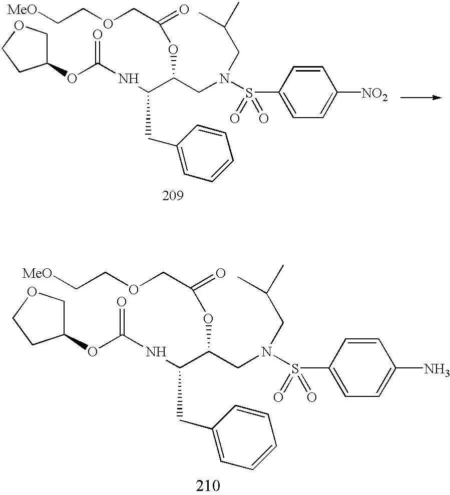

A slurry of 209 (170 mg) and 10 mg of 10% Pd.C in 95% EtOH was flushed with hydrogen in a scintillation vial equipped with septum and a stir bar. Continuous overnight hydrogenolysis under hydrogen balloon resulted in a complete conversion. The crude preparation was then filtered off the catalyst, and purified on RP C18 HPLC (Prep Nova-Pack C186 um, 60 A, gradient 0-100% B over 30 min. The desired product was collected and lyophilized affording a white fluffy solid (50 mg, 30.8%).

### EXAMPLE 13

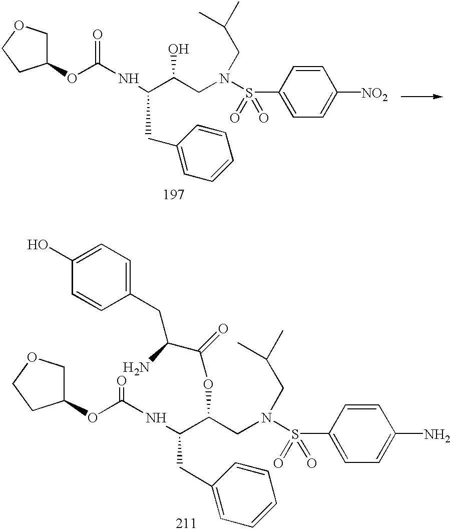

Compound 211 was obtained following the acylation and reduction procedures of Example 12. ES+669.2 (M+1), tHPLC=8.06 min (D), 13C NMR (DMSO)168.9, 156.9, 155.7, 153.1, 138.1, 130.5, 129.2, 129.1, 128.1, 126.2, 124.7, 122.5, 112.8, 76.2, 74.5, 72.5, 66.1, 58.0, 53.6, 52.6, 49.2, 33.6, 32.1, 26.6, 25.3, 20.0. tHPLC=11.71 min (D), ES+ 967 (M+1).

### EXAMPLE 14

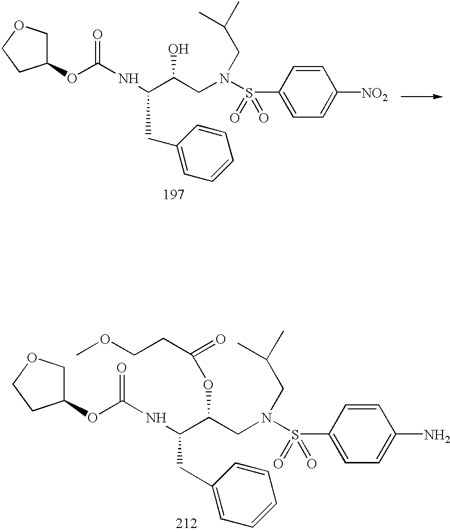

212 was obtained following the procedures of Example 12.

tHPLC=9.45 min (D), ES+592.2 (M+1).

13C NMR (DMSO) 171.5, 155.8, 148.9, 137.8, 129.5, 129.3, 128.5, 126.7, 115.2, 75.2, 73.8, 73.1, 68.3, 67.0, 58.7, 57.1, 53.3, 49.2, 35.4, 32.4, 26.7, 20.1, 19.8.

1H(CDCl3, 399.42 KHz): 8.33 (2H, d, J=8.8), 7.95 (2H, d, J=8.8), 7.23 (5H, m) 5.22 (m, 2H), 5.08 (m, 1H), 4.08 (m, 1H), 3.80-3.45 (7H, m), 3.41 (3H, s), 2.98 (m, 3H), 2.66 (m, 1H), 2.57 (m, 2H), 2.10 (s, 1H), 1.93 (2H, m), 0.82 (3H, d), 0.78 (3H, d).

ES+ 622 (M+1), 644 (M+Na) tHPLC=10.29 min (D).

13C NMR (CDCl3): 171.3, 155.5, 149.9, 145.6, 136.9, 129.2, 128.6, 128.5, 126.8, 124.4, 76.7, 75.3, 73.2, 72.9, 68.2, 66.9, 58.7, 55.9, 53.1, 48.3, 35.3, 32.7, 26.3, 19.9, 19.8.

### EXAMPLE 15

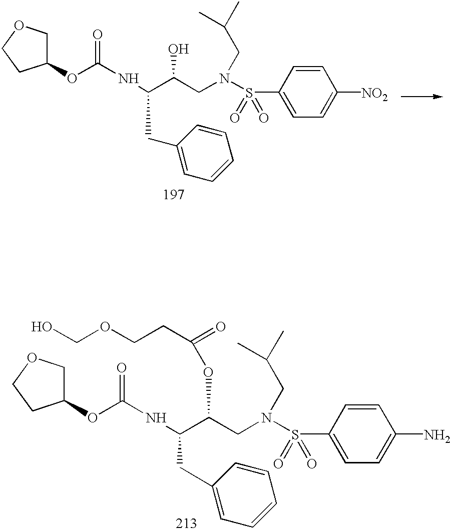

213 was obtained following the procedure of Example 12. tHPLC=9.21 min (D); ES+ 622 (M+1).

13C NMR (CDCl3): 170.54, 156.2, 148.6, 136.8, 129.4, 129.2, 128.6, 126.6, 115.7, 76.7, 74.6, 73.2, 71.8, 70.6, 68.2, 66.9, 58.9, 57.3, 53.8, 49.4, 36.2, 33.1, 26.8, 19.8, 19.5.

Intermediate: t HPLC=10.05 min (D); ES+=652 (M+H) 674 (M+Na).

### EXAMPLE 16

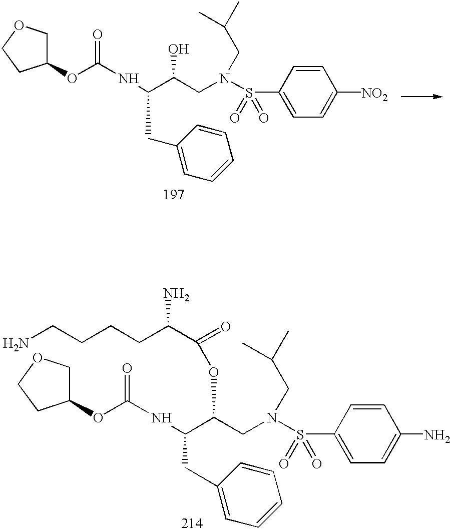

214 was obtained following the procedure of Example 12.

ES+ 634.4 (M+1); t HPLC=7.17 min (D).

13C (DMSO): 169.3, 155.8, 153.1, 138.0, 129.1, 129.0, 128.1, 126.3, 122.6, 112.8, 94.3, 75.6, 74.6, 72.4, 66.1, 57.8, 52.7, 52.0, 49.3, 38.4, 34.7, 32.2, 29.1, 26.6, 21.4, 20.1, 20.0.

### EXAMPLE 17

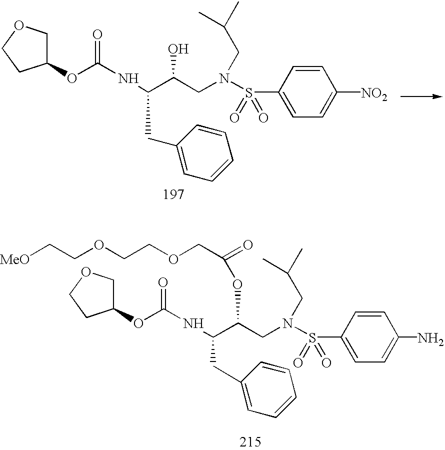

215 was obtained following the procedure of Example 12.

t HPLC=9.12 min (D)

1H (DMSO) all signals broad: 7.38 (3H, br m), 7.20 (5H, br m) , 6.62 (2H, br m), 5.15 (1H, br m), 4.92 (1H, br m), 4.00 (3H, m), 3.7-3.0 (16H, m), 2.78 (2H, m), 2.57 (3H, m), 2.04 (m, 1H), 1.78 (m, 2H), 0.77 (6H, m)

13C (DMSO) 170.6, 156.3, 153.7, 139.1, 129.8, 128.4, 126.7, 123.7, 113.3, 79.8, 79.2, 77.3, 76.1, 75.4, 75.2, 73.0, 71.9, 52.3, 51.8, 48.2, 46.7, 39.9, 38.7, 25.8, 22.6.

Intermediate:

t HPLC=10.18 min (D); ES+ 696.3 (M+1).

### EXAMPLE 18

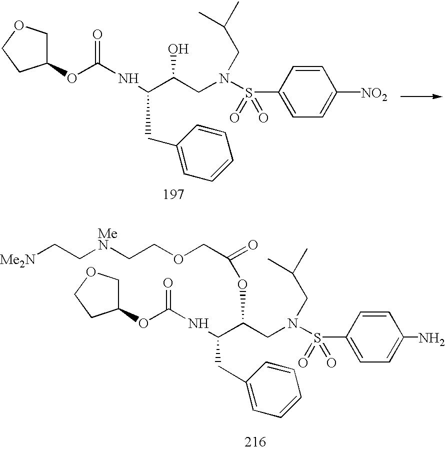

216 was obtained following the procedure of Example 12.

1H-NMR: 0.97 (6H,t), 1.95 (2H,m), 2.20 (1H,m), 2.9 (2H,m), 2.96 (6H,s), 3.00 (3H,s), 3.38 (1H,m), 3.42 (3H,m), 3.36 (1H,m), 3.6 (2H,m), 3.7 (6H,m), 3.98 (2H,m), 4.2 (2H,dd), 5.1 (1H,bs), 5.4 (1H,m), 6.8 (2H,d), 7.4 (5H,m), 7.6 (2H,d).

LC-MS: 1 peak, 692 (MH+).

### EXAMPLE 19

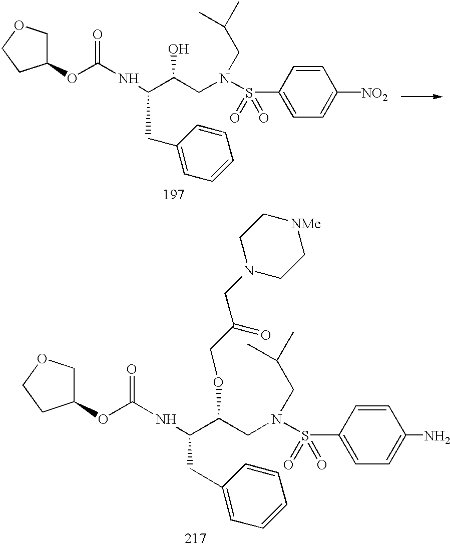

217 was obtained following the procedure of Example 12.

1H-NMR (CDCl3): 0.78 (6H,dd), 1.9 (2H,m), 2.1 (1H,m), 2.3 (3H,s), 2.9 (8H,m), 2.9 (2H,m), 3.15 (1H,m), 3.35 (1H,m), 3.5 (1H,m), 3.75 (4H,m), 4.06 (2H,s), 4.15 (2H,m), 4.9 (1H,dd), 5.05 (1H,bs), 5.2 (1H,bs), 6.63 (2H,d), 7.2 (5H,m), 7.55 (2H,d), 8.0 (2H,m).

ESMSP: 676 (MH+).

### EXAMPLE 20

### General Procedure for N-acylated Compounds

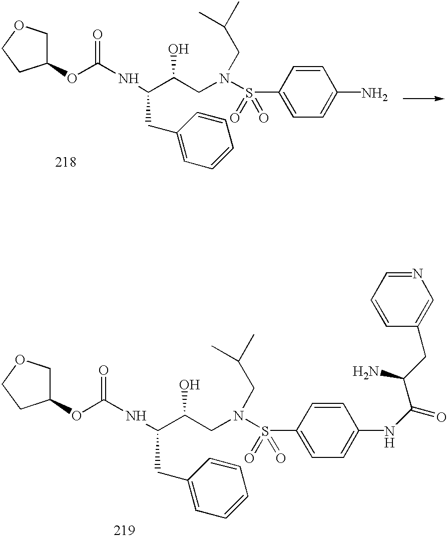

A mixture of 0.5 g (1 mMol) of (3S)-Tetrahydro-3-furfuryl-N-((1S,2R)-1-benzyl-2-hydroxy-3-(N-isobutyl-4-aminobenzenesulfonamido)propyl) carbamate, 0.4 g (1.5 mMol) of Boc-(S)-3-pyridyl alanine, 0.29 g (1.5 mMol) EDCI and 0.1 g 4-dimethylamino pyridine in 10 ml of N,N-dimethylformamide was stirred at 25° for 12 hours. The volatiles were removed in vacuo and the residue was partitioned between ethyl acetate and 1N hydrochloric acid. The organic layer was washed with 1N sodium hydroxide and brine, dried over magnesium sulfate and concentrated in vacuo. The residue was chromatographed on a 2 inch plug of silica gel (1:1 ethyl acetate: hexane) to give the desired N-acylated material. Deprotection by treatment with 50 ml of trifluoroacetic acid, followed by co-evaporation of residual acid with methanol gave the desired prodrug as a white foam (0.2 g, 26%).

H1-NMR (acetonitrile-D3): 0.95 (6H,dd), 2.0 (2H,m), 2.25 (1h,m), 2.8-3.1 (5H,m), 3.6-4.0 (7H,m), 4.25 (1H,m), 4.75 (1H,m), 5.18 (1H,m), 5.45 (1H,m), 7.0 (2H,d), 7.4 (5H,m), 7.75 (2H,d), 8.2 (1H,m), 8.8 (1H,d), 8.85 (1H,d), 9.15 (1H,s).

LC/MS: 1 peak, 654 (MH+).

### EXAMPLE 21

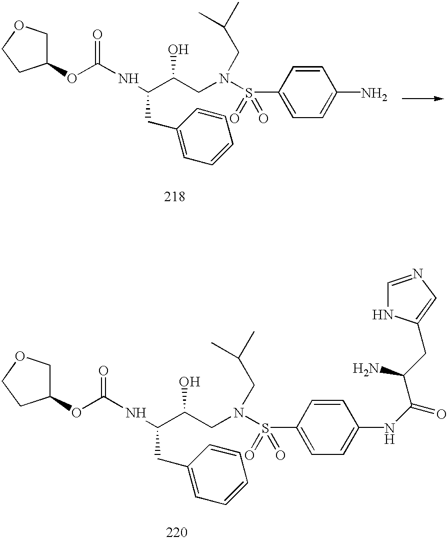

220 was obtained using the general procedure in Example 20.

1H-NMR (acetone-d6/methanol-d4): 0.95 (6H,t), 2.0 (2H,m), 2.2 (1H,m), 2.90 (1H,dd), 2.95 (2H,d), 3.12 (1H,dd), 3.4 (2H,m), 6 (1H,d), 3.8 (5H,m), 4.4 (2H,bm), 6.82 (2H,d), 7.20 (1H,s), 7.4 (5H,m), 7.65 (2H,d), 8.0 (1H,s).

LC/MS: 1 peak, 643 (MH+).

### EXAMPLE 22

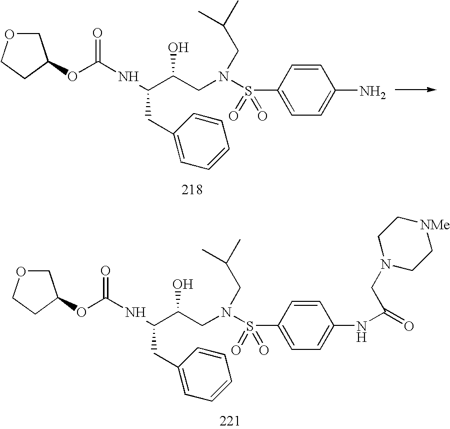

221 was obtained using the general procedure in Example 20.

1H-NMR (DMSO d-6): 0.76 (6H,t), 1.80 (2H,m), 2.10 (1H,m), 3.7 (4H,m), 3.75 (3H,s), 3.2 (5H,m), 3.58 (2H,s), 3.7 (4H,m), 4.97 (1H,bm), 5.18 (1H,bs), 6.7 (2H,d), 7.22 (5H,m), 7,45 (2H,d).

LC/MS: 1 peak, 646 (MH+).

### EXAMPLE 23

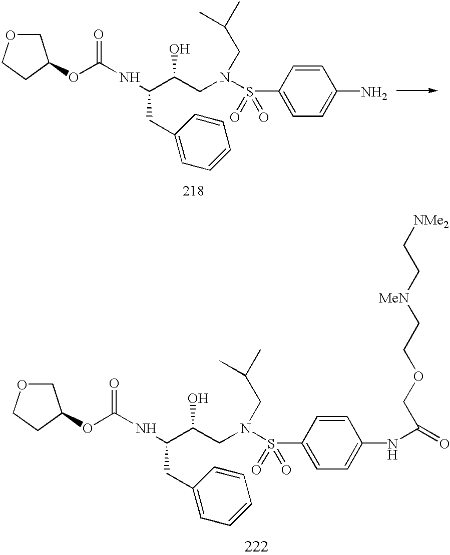

222 was obtained using the general procedure in Example 20.

1HNMR (acetonitrile d-3): 1.0 (6H,t), 2.0 (2H,m), 2.2 (1H,m), 3.00 (6H,s), 3.02 (3H,s), 3.1 (4H,m), 3.5 (3H,m), 3.8 (8H,m), 4.4 (2H,s), 5.15 (1H,bs), 7.4 (5H,m), 7.97 (2H, d), 8.04 (2H,d),

LC/MS: 1 peak, 692 (MH+).

### EXAMPLE 24

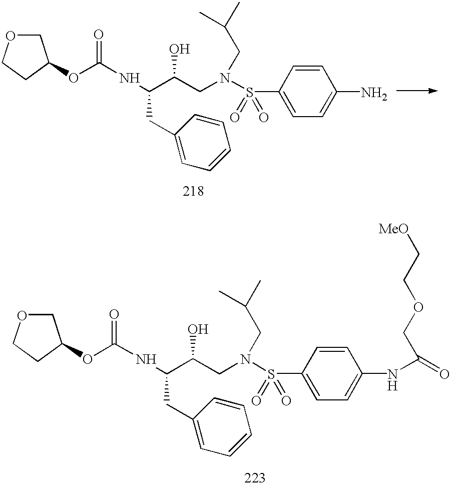

223 was obtained using the general procedure in Example 20.

t HPLC=9.22 min (D); ES+ 622 (M+1).

1H NMR d6-DMSO: 0.76 (6H,dd), 1.0-1.8 (15H,m), 2.03 (1H,m), 2.58 (2H,m), 2.79 (2H,m), 3.11 (1H,m), 3.28 (3H,s), 3.3-3.5 (12H,m), 3.94 (1H,m), 4.08 (1H,m), 4.94 (1H,m), 5.14 (1H,m), 6.61 (2H,d), 7.22 (5H,m), 7.40 (3H,m).

13C (DMSO)169.7, 165.9, 152.9, 138.4, 129.2, 129.1, 128.1, 126.2, 123.1, 112.8, 74.4, 74.1, 72.5, 71.2, 69.8, 66.1, 58.1, 57.1, 52.9, 47.5, 33.4, 33.2, 26.3, 24.5, 18.9, 18.8.

### EXAMPLE 25

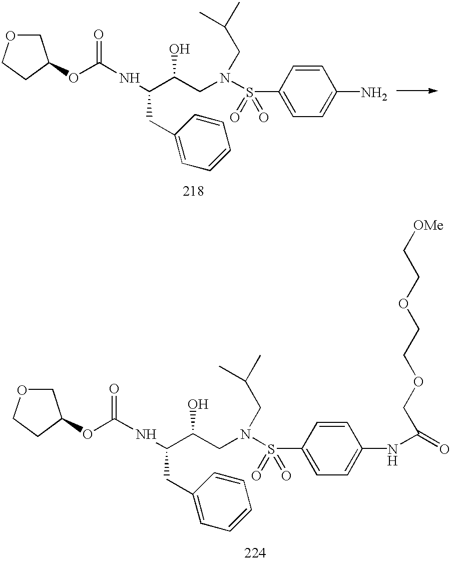

224 was obtained using the general procedure in Example 20.

### EXAMPLE 26

### O, N-diacylated Prodrugs

The general procedure for N,O-diacylated compounds followed the protocol outlined in Example 20, above, except that a five fold excess of reagents was used relative to the starting material.

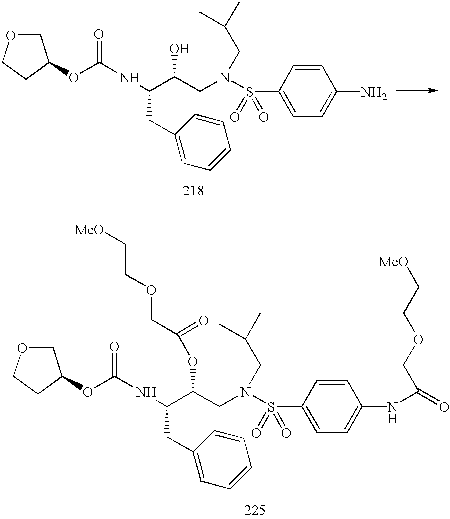

t HPLC 9.26 min (D); ES+ 738 (M+1) 760 (M+Na).

13C (DMSO): 170.2, 169.8, 156.4, 143.4, 138.8, 129.5, 128.8, 128.5, 126.8, 119.7, 74.9, 74.2, 73.7, 71.6, 70.7, 70.3, 68.0, 67.2, 59.3, 57.6, 53.8, 49.6, 35.7, 33.8, 27.1, 20.4.

1H (DMSO): 10.1 (1H, s), 7.84 (d, 2H, J=8.5), 7.76 (d, J=8.7, 2H), 7.40 (1H, d, J=9.2), 7.22 (m, 5H), 5.14 (1H, m), 4.95 (1H, m), 4.1 (m, 8H), 3.7-3.3 (m, 13H), 3.28 (s, 3H), 3.26 (s, 3H), 2.86 (m, 2H), 2.73 (m, 1H), 2.59 (m, 1H), 2.04 (m, 1H), 1.83 (m, 2H), 0.78 (m, 6H).

### EXAMPLE 27

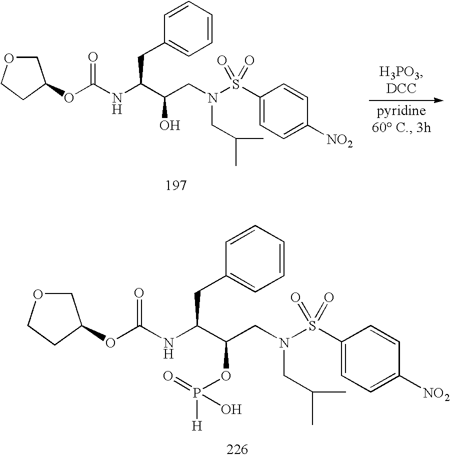

To a mixture of 197 (2.93 g, 5.47 mmol) and phosphorous acid (Aldrich, 2.2 equiv., 12.03 mmol, 987 mg) in 20 ml pyridine was added 1,3-dicyclohexylcarbodiimide (Aldrich, 2.1 equiv., 11.49 mmol, 2.37 g) and the reaction heated to 60° C. under nitrogen for 3 h. Solvent was removed in vacuo, the residue treated with 200 ml 0.1N aqueous sodium bicarbonate and stirred 1 h at ambient temperature. The mixture was filtered, the filtrate acidified to pH 1.5 by addition of conc. HCl and extracted with ethyl acetate (3×100 ml). The combined orgnic layers were dried over magnesium sulfate, filtered and concentrated in vacuo to give 3.15 g (96%) of desired product 226 which was used directly in the next reaction. HPLC: Rt=8.91 min (96%), MS (AP+) 600.5 (M+1).

### EXAMPLE 28

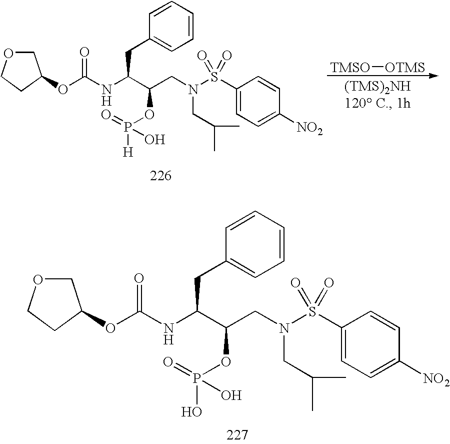

A suspension of 226 (˜5.47 mmol) in 18 ml hexamethyldisilazane was stirred at 120° C. until homogeneous followed by addition of bis(trimethylsilyl) peroxide (Gelest, Inc., 2.3 equiv., 12.58 mmol, 2.24 g, 2.71 ml). After 1 h the mixture was cooled to ambient temperature, solvent removed in vacuo, the residue stirred with 100 ml methanol, solvent removed in vacuo, the residue stirred with 100 ml 0.1N aqueous sodium bicarbonate, acidified to pH 1.5 by addition of conc. HCl, saturated with brine and extracted with ethyl acetate (3×100 ml). The combined organic layers were dried over magnesium sulfate, filtered and concentrated in vacuo to give 2.98 g (88%) of desired product 227, which was used directly in the next reaction. HPLC: Rt=9.28 min (90%), MS (AP+) 616.5 (M+1).

Alternatively, 227 can be synthesized directly from 197. In this method, 197 was dissolved in pyridine (300 mL). The resulting solution was concentrated in vacuo to about 150 ml at 50-55° C. The solution was then cooled under N₂ to 5° C., and treated with POCl₃ (6.5 ml, 1.24 equiv.) over 2 minutes. The cooling bath was removed and the reaction stirred at ambient temperature for 2.5 hrs. The solution was then cooled to 5° C. and water (300 ml) was added over 30 minutes.

The resulting mixture was extracted with 4-methylpentan-2-one (MIBK, 2×150 ml). The combined extracts were washed with 2N HCl (2×250 ml). The acid washes were back extracted with MIBK (60 ml), then the combined MIBK solutions were treated with 2N HCl (150 ml). The two phase mixture was stirred rapidly and heated to 50° C. for 2 hours. The reaction mixture was cooled to 20° C., the phases were separated and the MIBK solution was washed with brine (150 ml). The product, 227, was isolated by drying the solution with magnesium sulfate, filtering of the drying agent and concentrating in vacuo at 40° C. to give the product as a pale yellow foam (31 g, 90% yield)

### EXAMPLE 29

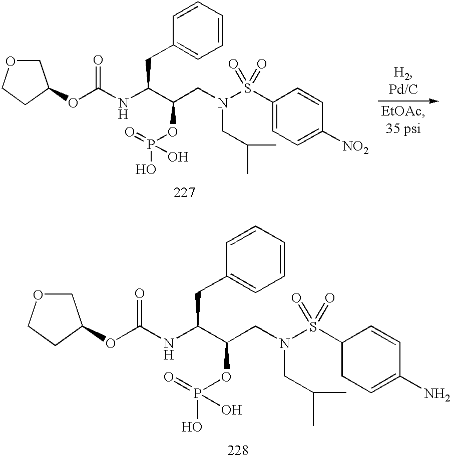

A solution of 227 (2.98 g, 4.84 mmol) in 50 ml ethyl acetate was treated with 10% palladium on carbon (Aldrich, 300 mg) and put under 35 psi of hydrogen on a Parr shaker for 15 h. Catalyst was removed by filtration and solvent removed in vacuo to give 2.66 g (94%) of desired product 228. HPLC: Rt=7.23 min (92%), MS (ES+) 586.3 (M+1).

### EXAMPLE 30

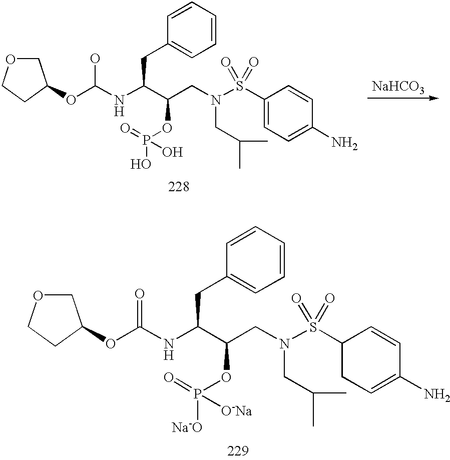

Solid 228 (2.66 g, 4.54 mmol) was treated with 10 ml aqueous sodium bicarbonate (Baker, 3.0 equiv., 13.63 mmol, 1.14 g) and loaded onto a resin column (Mitsubishi Kasei Corp., MCI-gel, CHP-20). Distilled water was run through until the eluent was neutral followed by product elution with 1 acetonitrile in water. Pure fractions were pooled and lyophilized to give 918 mg of pure bis-sodium salt 229.

### EXAMPLE 31

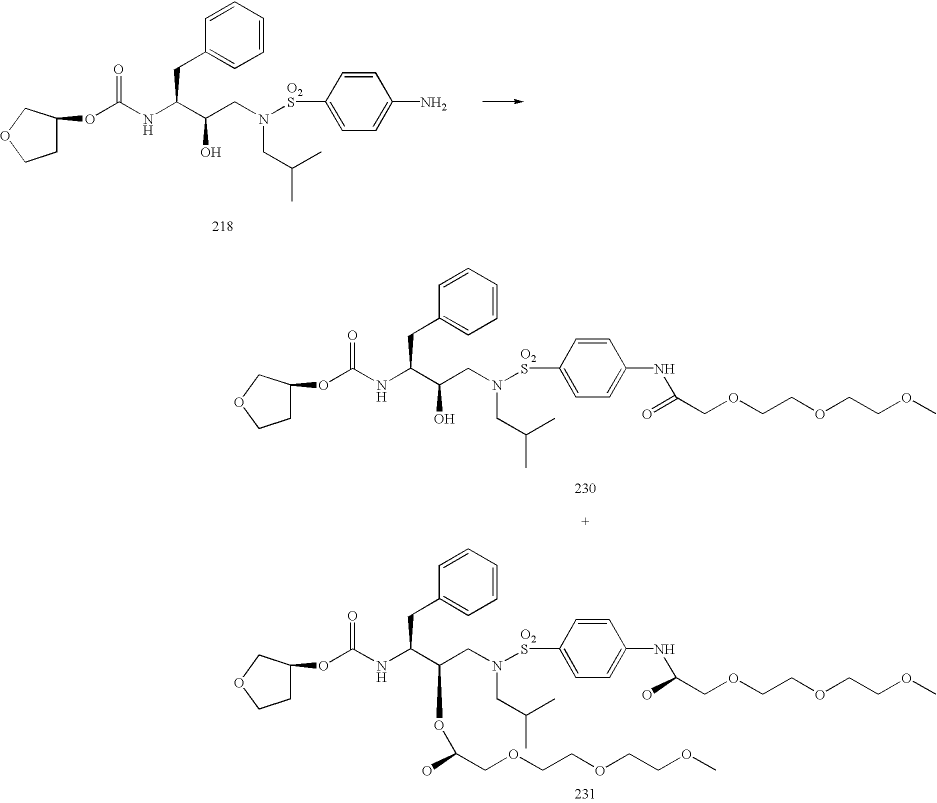

0.53 g (3.0 mmol) 2-[2-(2-Methoxyethoxy)ethoxy] acetic acid was added to a stirred solution of 1.2 g (3.15 mmol) HATU 0.2 g (1.47 mmol) HOAt 0.4 g (4.0 mmol) NMM in 10 ml anhydrous N,N-dimethylformamide. The mixture was stirred at room temperature for 30 minutes, then 0.5 g (1 mmol) of (3S)-Tetrahydro-3-furfuryl-N-((1S,2R)-1-benzyl-2hydroxy-3-(N-isobutyl-4-aminobenzenesulfonamido)-propyl) carbamate was added to the solution in one portion. The mixture was stirred at 20° C. for an hour then at 50° C. for an additional 12 hours. It was then cooled to 20° C., 50 ml of ether was added, and the solution was washed with water three times. The aqueous phase was washed with ether, and then the combined organic phases were dried with anhydrous magnesium sulfate and filtered. The filtrate was concentrated under reduced pressure and the residue was purified by silica gel chromatography to obtain the desired Mono-(N)acylated (102 mg, 15%) and Bis-(O,N) acylated (262 mg, 32%) compounds.

Mono-(N)-acylated: 1H-NMR(CDCl3): 0.85 (dd, 6H), 1.85 (m, 2H), 2.08 (m,1H), 2.8-3.1 (m, 7H), 3.33 (s, 3H), 3.55 (m, 3H), 3.70-3.90 (m, 8H), 4.1 (s, 2H), 5.0 (d, 1H), 5.08 (s(br), 1H), 7.2 (m, 5H), 7.70 (d, 2H), 7.80 (d, 2H), 9.09 (s, 1H).

MS(FAB+) : 666 (M+1).

Bis-(O,N)-acylated: 1H-NMR(CDCl3): 0.77 (m, 6H), 1.81 (m, 1H), 1.95 (m, 1H), 2.05 (m, 1H), 2.6-3.0 (m, 6H), 3.2 (m,1H), 3.332 (s, 3H), 3.338 (s, 3H), 3.5-3.8 (m, 18H), 4.1 (s, 2H), 4.14 (s, 2H), 4.17 (m, 1H), 5.05 (m, 2H), 5.25 (s(br), 1H), 7.2 (m,5H), 7.69 (d, 2H), 7.78 (d 2H), 9.06 (s, 1H).

MS(FAB+): 826(M+1), 848(M+Na).

### EXAMPLE 32

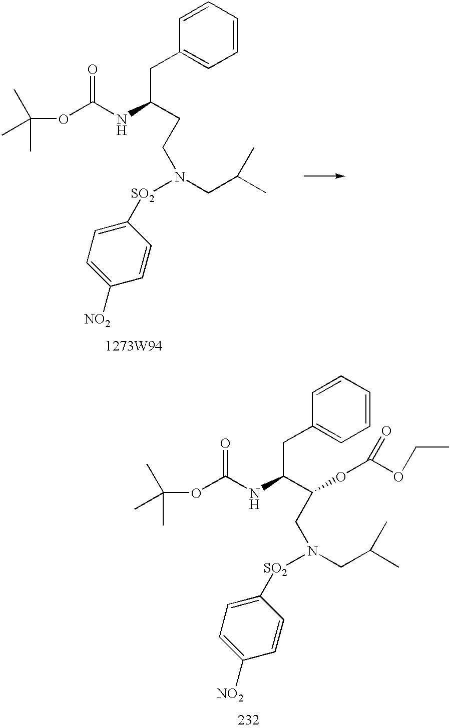

We dissolved 0.521 g (1 mM) of 1273W94 in 5 ml THF, then cooled to −78° C. under nitrogen, and added 1.56 ml (2.5 mM) of a 1.6 M solution of nBuLi in hexane. After 20 min at −78° C., we added 105 μL (1.1 mM) of ethyl chlorocarbamate and warmed up the reaction to room temperature, followed by addition of another 105 μL of ethyl chlorocarbamate.

After stirring for additional 4 hrs, the reaction was quenched with water and the organic solvent evaporated. Part of the crude product was purified on a silica gel (Rf=0.69 (1:2 ethyl acetate:hexane)), yielding 0.131 g of the product.

C,H,N: calc: 46.06, 4.97, 5.88, found 45.90, 4.97, 5.88 C₂₃H₃₃N₅O₅S₁. 2.2 TFA

LC/MS (ES+) 594 (M+1) 1 peak at 6.96 min.

Analytical HPLC(A) t=24.57 min.

13C (CDCl3): 155.8, 154.4, 149.9, 145.7, 136.8, +129.2, +128.7, +126.8, +124.2, 80.1, +76.9, −64.3, −56.2, −52.5, −48.7, −36.2, +28.1, +26.4, +20.0, +19.8, +14.3.

### EXAMPLE 33

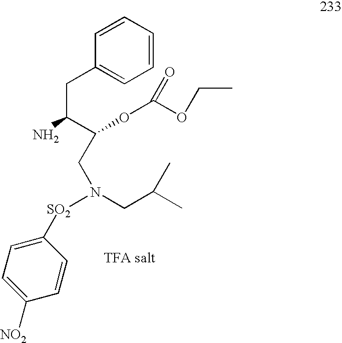

We dissolved 0.131 g of the above ethyl carbonate in 4 ml DCM, followed by 4 ml of TFA. Solvents were then removed after 45 min at room temperature, resulting in the title compound.

1H (DMSO): 8.37 (2H, d, J=7.2), 8.15 (2H, m), 8.00 (2H, d, J=7.0), 7.37 (5H, m), 5.04 (1H, d, J=6.9), 4.06 (2H, q, J=7.0), 3.82 ((1H, m), 3.35 (2H, m), 2.95 (4H, m), 1.82 (1H, m), 1.20 (3H, t, J=7.0), 0.72 (overlapping doublets, 6H, J=6.2).

LC/MS 1 peak at 4.76 min.

ES+ 497.3 (M+1).

### EXAMPLE 34

### O, N-Acyloxy Rearrangement

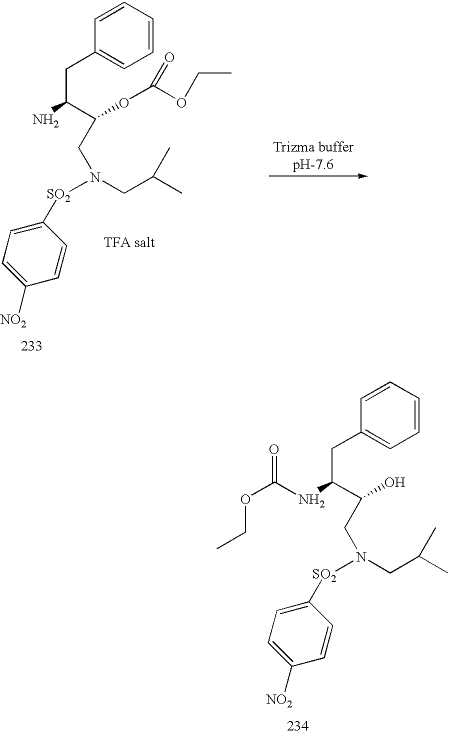

C,H,N: calc:53.26, 6.14, 7.57, found 53.22, 6.14, 7.57 C₂₃H₃₃N₅O₅S₁×0.8 TFA

LC/MS (ES+) 594 (M+1) 1 peak at 6.96 min.

Analytical HPLC(A) t=24.57 min.

1H (DMSO): 8.34 (2H, d, J=8.7), 8.02 (2H, d, J=8.0), 7.19 (5H, m), 6.98 (1H, d, J=7.2), 5.00 (1H, m), 3.83 (2H, q), 3.50 (2H, m), 3.06 (m, 2H), 2.96 (2H, m), 2.43 (1H, m), 1.97 (1H, m), 1.02 (3H, t), 0.84 (3H, d), 0.82 (3H, d). 13C (DMSO): 156.2, 150.1, 145.7, 140.0, +129.7, +129.2, +128.5, +126.3, +125.0, +71.8, −60.0, +56.2, −56.0, −51.8, −36.0, +26.3, +20.3, +20.1, +14.6.

### EXAMPLE 35

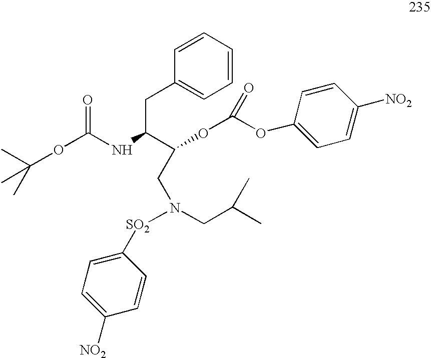

Synthesis of 235 was accomplished analogous to that set forth in Example 1.

Yield 15.2%; tHPLC=25.2 min (A).

Rf=0.54 (B); ES+ 687.3 (M+1).

1H (CDCl3): 8.34 (overlapping d+d, 4H), 7.97 (d, 2H, J=8.9), 7.35 (7H, m), 5.09 (1H, m), 4.56 (1H, d, J=8.4), 4.20 (1H, m), 3.54 (1H, m), 3.00 (3H, m), 2.82 (1H, m), 1,84 (1H, m), 1.37 (9H, s), 0.84 (3H, d), 0.82 (3H, d).

### EXAMPLE 36

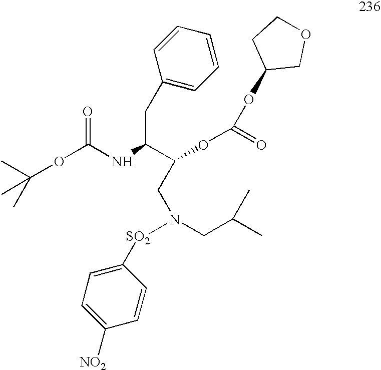

We dissolved 150 mg of 235 in 3 ml of anhydrous dioxane, added 0.35 ml of S(+)-3-OH-THF and 0.14 ml triethyl amine. The mixture was refluxed gently under nitrogen for 2 days. Conversion to 236 was quantitative. Solvents were removed and the compound purified on silica (B).

tHPLC=22.98 min (A); ES+ 636.2 (M+1).

1H NMR (CDCl3): 8.29 (2H, d), 7.91 (2H, d), 7.22 (5H, m), 5.13 (1H, m), 4.96 (1H, m), 4.52 (1H, d), 4.02 (1H, m), 3.84 (2H, m), 3.44 (1H, m), 3.36 (1H, m), 3.10 (3H, m, overlap), 2.88 (2H, m), 2.64 (1H, m), 2.14 (1H, m), 2.05 (1H, m), 1.84 (1H, m), 1.27 (9H, s), 0.78 (6H, two overl. d).

### EXAMPLE 37

### Carbohydrate-Based Prodrugs

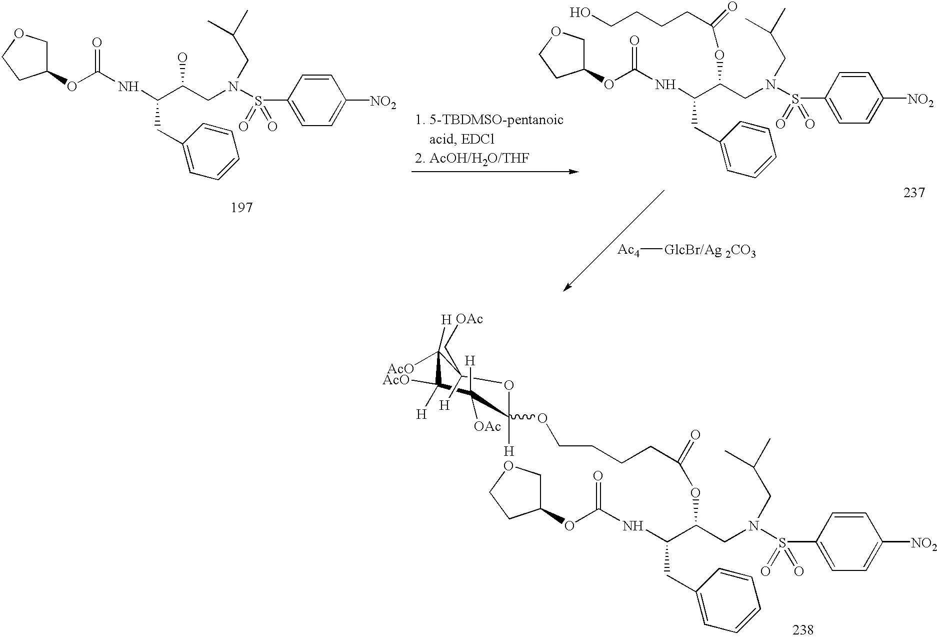

A mixture of 0.54 g (1 mMol) of (3S)-Tetrahydro-3-furfuryl-N-((1S,2R)-1-benzyl-2-hydroxy-3-(N-isobutyl-4-aminobenzenesulfonamido)propyl) carbamate, 0.46 g (2 mMol) of 5-dimethyl-tert-butyosilyloxypentanoic acid, 0.346 g (1.8 mMol) of EDCI and 0.556 mL (4 mMol) of triethylamine in 10 ml of dimethyl formamide was stirred at rt for 24 h. Another 3 mMol each of acid, EDCI and triethylamine were added and stirring was continued for an additional 96 h. A third batch of acid and EDCI was added (3 mMol each) and the mixture was stirred 72 h to complete the reaction.

The reaction mixture was then diluted with ethyl acetate and extracted with 1 N hydrochloric acid, saturated sodium bicarbonate and water. Evaporation of the solvent and purification on silica gel (30% ethyl acetate-hexane) gave the desired product (500 mg) as a waxy solid.

LCMS: 1 peak, 772.5 (M+Na)

1H NMR (CDCL3): 0.01 (6H,s), 0.78 (6H,dd), 0.95 (9H,s), 1.4-1.8 (6H,m), 1.9 (2H,m), 2.05 (1H,m), 2.3 (2H,m), 2.65 (1H,m), 2.95 (2H,m), 3.22 (1H,m), 3.4 (1H,m), 3.6 (2H,m), 3.75 (3H,m), 4.8 (1H,d), 5.1 (1H,bs), 5.2 (1H,bs), 7.2 (5H,m), 7.95 (2H,d), 8.36 (2H,d).

450 mg of the 238 was dissolved in 30 ml of tetrahydrofuran and treated with 20 ml of water and 50 ml of acetic acid. The mixture was stirred at rt for 2 h and evaporated. Titration with hexane gave the desired alcohol (290 mg) as a white solid.

A mixture of 0.15 g (0.24 mMol) of the alcohol produced above from the previous reaction, 0.205 g (0.5 mMol) of tetraacetylglucosylbromide and 0.191 g (0.7 mMol) of silver carbonate in 3 ml of dichloromethane was stirred at rt for 6 h. 150 mg of additional glucosyl bromide and 150 mg of silver carbonate were added and the mixture was stirred at rt overnight. The mixture was loaded onto a pad of silica gel and eluted with 30% ethylacetate-hexane to afford the desired protected carbohydrate pro-drug as a white foam (200 mg).

LCMS: 1 peak, 966 (M+H).

1H-NMR (CDCl3): 0.78 (6H,dd), 1.9 (2H,m), 2.00 (3H,s), 2.02 (3H,s), 2.05 (3H,s), 2.06 (3H,s), 2.1 (2H,m), 2.3 (2H,m), 2.7 (1H,m), 2.94 (3H,bd), 3.35 (2H,m), 3.45 (2H.m), 3.8 (5H,m), 4.1 (3H,m), 4.5 (1H,d), 4.9 (1H,bs), 4.95 (1H,t,), 5.08 (4H,m), 2H,d), 8.35 (2H,d).

### EXAMPLE 38

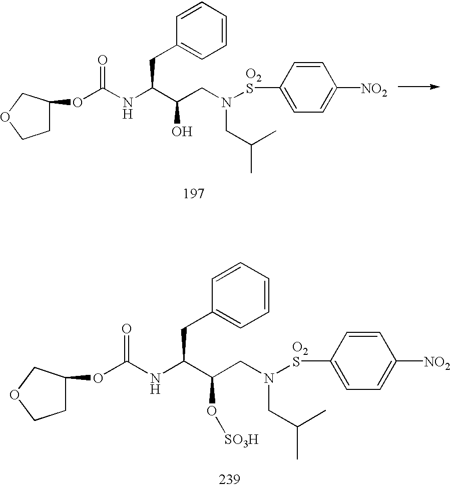

1.5 g (9.4 mmol) SO3.py complex was added to a stirred solution of 1 g (1.87 mmol) of 197 in 25 mL anhydrous tetrahydrofurane. The mixture was stirred at 20° C. for 12 hours, then filtered. The filtrate was concentrated at reduced pressure, and the residue was transferred to a silica gel column and eluted with EtOAc (neat), followed by EtOAc:EtOH (4:1) to obtain 471 mg (47%) 239 as a colorless foam.

1H-NMR(CDCl3): 0.80 (m, 6H), 1.8-2.1 (m, 3H), 4.15 (s(br), 1H), 4.8 (t, 1H), 5.04 (s (br), 1H).

MS (ES−): 614 (M−1)

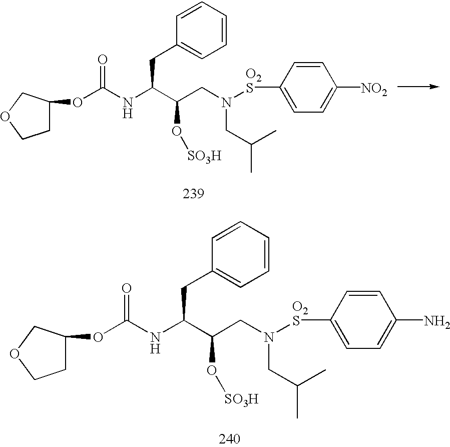

100 mg (0.162 mmol) 239 dissolved in 15 ml anhydrous tetrahydrofuran and 200 mg Pd/BaSO4 (5%) was added to the solution. The mixture was stirred under atmospheric pressure of hydrogen for 8 hours, and then the catalyst was filtered. The filtrate was concentrated under reduced pressure then dried under vacuum (˜1 Hg mm, 48 hrs.) to produce 80 mg (81%) 240 as a colorless foam. 1H-NMR(DMSO-d6): 0.85 (dd, 6H), 0.90 (m, 1H), 2.05 (m, 2H), 2.58 (m, 3H), 2.84 (dd, 1H), 3.05 (m, 2H), 3.55-3.80 (m, 6H), 4.20 (t, 1H), 4.42 (m, 1H), 4.93 (s(br), 1H), 6.09 (s, 2H), 6.70 (d, 2H), 6.80 (d, 1H), 7.15-7.40 (m, 4H), 7.51 (d, 2H).

MS(ES−): 584 (M−1).

### EXAMPLE 39

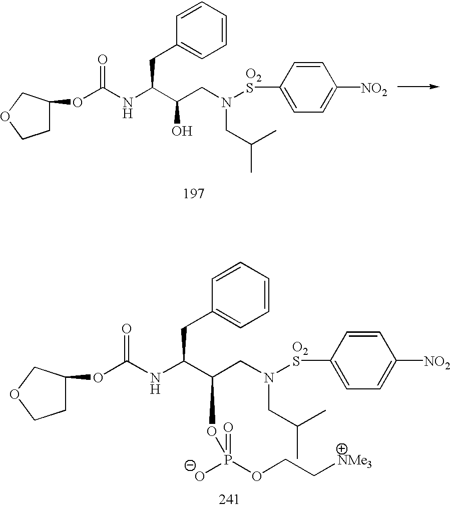

780 mg (3 mmol) 2-Chloro-1,3,2-dioxaphospholane was added to a stirred solution of 1.07 g (2 mmol) 197 and 0.7 ml (4 mmol) N,N-Diisopropylethylamine in 25 ml dichloromethane at 0° C. The mixture was allowed to warm up to room temperature and it was stirred for 2 hours. The mixture was then cooled to 0° C. and 1.5 g (9.3 mmol) bromine was added in 5 ml dichloromethane. The mixture was stirred for 1 hour at 20° C., followed by evaporation under reduced pressure. An aqueous solution (50%) of 15 ml trimethylamine was added to the residue, and the mixture was stirred at 20° C. for 12 hours.

Solvents were removed under reduced pressure and 50 ml EtOAc:EtOH (9:1) was added to the residue. The solid was filtered, washed with EtOAc:EtOH (9:1) then the filtrate was concentrated under reduced pressure. The residue was chromatographed on a 3 inch plug of silica gel using ethyl acetate (neat), then methanol (neat), as eluents to obtain 1.15 g (82%) 241 as an off-white solid.

1H-NMR(CDCl3): 0.60 (dd, 6H), 1.70 (m, 1H), 1.95 (m, 1H), 2.10 (m, 1H), 2.8-3.2 (m, 6H), 3.4 (s (br), 9H), 5.09 (s(br), 1H), 7.25 (m, 5H), 7.83 (d, 2H), 8.28 (d, 2H). MS(ES+): 701 (M+1), 184 (phosphatidyl choline+).

### EXAMPLE 40

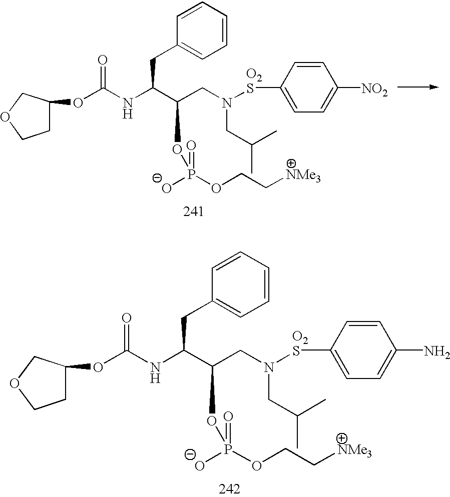

250 mg PdC (10%) was added to a solution of 250 mg (0.35 mmol) 241 in 10 ml methanol, and the mixture was stirred under atmospheric pressure of hydrogen for 4 hours at 20° C. The mixture was filtered, and the filtrate was concentrated under reduced pressure. The residue was then dissolved in 10 ml water and lyophilized to obtain 174 mg (74%) 242 as white solid.

1H-NMR(DMSO-d6): 0.82 (dd, 6H), 1.80-2.00 (m, 2H), 2.10 (m, 1H), 2.80 (m, 3H), 3.00 (m, 2H), 3.2 (s (br), 9H), 4.0-4.3 (m, 4H), 4.91 (s(br), 1H), 6.08 (s(br), 2H), 6.67(d, 2H), 7.30 (m, 5H), 7.48 (d, 2H), 8.12 (d, 1H).

MS(ES+): 671 (M+1), 184 (phosphatidyl choline+).

### EXAMPLE 41

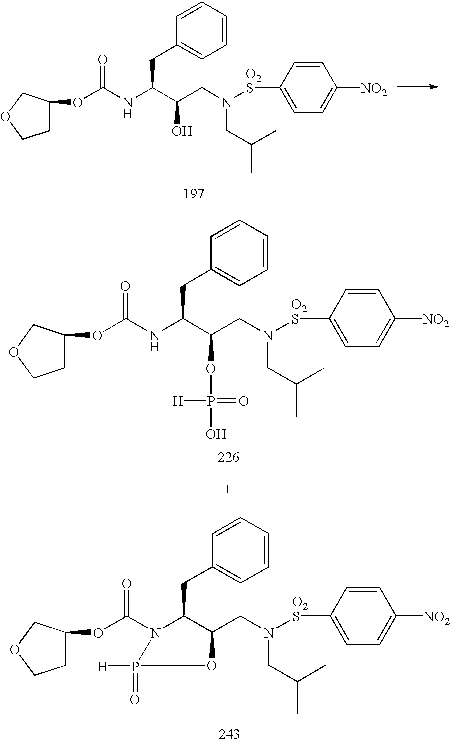

0.175 ml (2 mmol) phosphorus trichloride was added to a stirred solution of 1.07 g (2 mmol) 197 and 0.35 ml (2 mmol) N,N-Diisopropylethylamine in 25 ml dichloromethane at 20° C. The mixture was stirred for 4 hours at 20° C., then 1 ml water was added and stirred for an additional 12 hours at 20° C. 3 g anhydrous magnesium sulfate was added to the mixture and it was stirred for 30 minutes, then filtered. The filtrate was concentrated under reduced pressure and purified by silica gel chromatography using EtOAc:Hexane (4:1), then EtOAc:EtOH (1:1), to obtain 402 mg (48%) 226 and 427 mg (36%) 243. 226:

1H-NMR(DMSO-d6): 0.82 (dd, 6H), 1.84 (m, 1H), 1.98 (m, 1H), 2.10 (m, 1H), −2.68 (dd, 1H), 2.9-3.2 (m, 4H), 3.6-3.8 (m, 3H), 3.94 (t, 1H), 4.30, (s(br), 1H), 4.97 (s(br), 1H), 7.30 (m, 5H), 8.14 (d, 2H), 8.43 (d, 2H).

MS(ES−): 598 (M−1).

243: (1:1 mix of diastereomers):

1H-NMR(CDCl3): 0.80 (m, 6H), 1.8-2.1 (m, 4H), 2.8-3.2 (m, 6H), 3.7-3.9 (m, 4H), 4.15 (m, 1H), 4.8-5.15 (m, 2H), 5.57, 5.72 ((d,d), 1H), 7.25 (m, 5H), 7.95 (dd, 2H), 8.35 (m, 2H).

MS(ES−): 580 (M−1), 598 ((M+H2O)−1).

### EXAMPLE 42

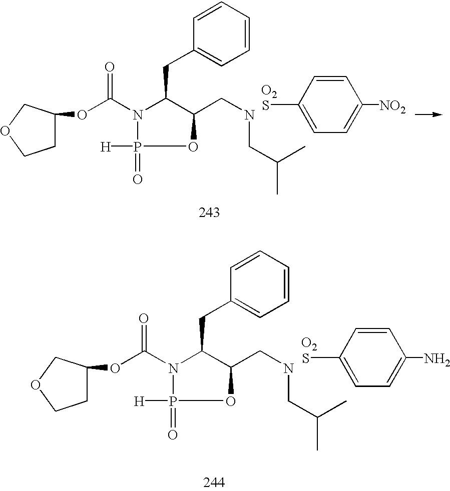

The reduction was carried out as described in Example 40; (Yield: 79%).

1H-NMR(DMSO-d6): 0.81 (dd, 6H), 1.82 (m, 1H), 1.95 (m, 1H), 2.08 (m, 1H), 2.6-3.15 (m, 6H), 3.6-3.75 (m, 3H), 4.03 (t, 1H), 4.28, (m, 1H), 4.96 (s(br), 1H), 6.07 (s, 2H), 6.65 (d, 2H), 7.25 (m, 5H), 7.42 (d, 2H).

MS (ES−): 568 (M−1).

### EXAMPLE 43

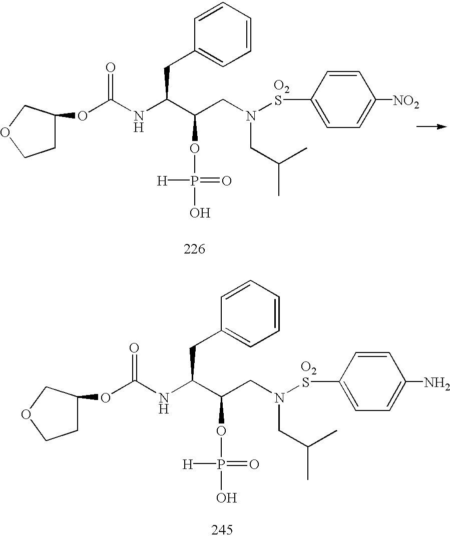

The reduction was carried out as described in Example 40; (Yield; 98%).

(1:1 mix of diastereomers):

1H-NMR(DMSO-d6): 0.82 (m, 6H), 1.75-2.0 (m, 2H), 2.05 (m, 1H), 2.6-3.2 (m, 6H), 3.55-3.8 (m, 4H), 4.02, 4.22 (m, t, 1H), 4.75 (m, 1H), 4.90, 5.01 ((d,d), 1H), 6.12 (s, 1H), 6.68 (d, 2H), 7.30 (m, 5H), 7.49 (d, 2H).

MS(ES−): 550 (M−1), 568 ((M+H20)−1).

### EXAMPLE 44

### Pharmacokinetics In Sprague-Dawley Rats Following Single Oral Dose

In order to study the pharmacokinetics of the prodrugs of this invention, we administered single oral doses of a series of prodrugs of this invention, as well as VX-478, to male and female Sprague-Dawley rats. Administration of molar equivalents of a series of prodrugs of this invention in a variety of pharmaceutical vehicles was tested.

Separate groups of male and female Sprague-Dawley rats (3/sex/group) received oral doses of compound 229 by oral gavage, in different vehicles at the same dose equivalent (40 mg/kg molar equivalent of VX-478). The different vehicles for compound 229 were: 1) water; 2) 5/4/1; 3) PEG 400; 4) TPGS/PEG 400; and 5) PEG. The vehicles for VX-478 were: 1)33% TPGS/PEG400/PEG; and 2) 12.5% TPGS/PEG 400/PEG.

Blood samples were collected following administration at various time intervals and analyzed for the presence of both compound 229 and its metabolite, VX-478, by HPLC and MS methods. The results of this study are tabulated below (Table IV).

TABLE IV

| Compound | 229 | 229 | 229 | 229 | VX-478 | VX-478 |
|---|---|---|---|---|---|---|
| vehicle | H₂O | H₂O:PG:EtOH | PEG | TPGS/PEG | 33% | 12.5% |
| | | 5:4:1 | 400 | 400/PG | TPGS/ | TPGS/ |
| | | | | | PEG | PEG |
| | | | | | 400/PG | 400/PG |
| number | 3 | 3 | 3 | 3 | 6 | ≧3 |
| of rats | | | | | | |
| Molar | 40 PO | 40 PO | 40 PO | 40 PO | 41 PO | 50 PO |
| equiv. | | | | | | |
| dose/ | | | | | | |
| 478 Dose | | | | | | |
| (mg/Kg) | | | | | | |
| AUC | 11.7 ± | 10.6 ± | 7.4 ± | 8.2 ± | 29.6 ± | 16.2 ± |
| (ug*hr/m | 4.8 | 7.4 | 1.8 | 1.6 | 5.8 | 1.8 |
| 1) | | | | | | |
| Cmax | 7.1 ± | 3.3 ± | 3.1 ± | 3.0 ± | 14.0 ± | 6.0 ± |
| (μM) | 1.7 | 0.6 | 0.3 | 0.7 | 2.2 | 1.0 |
| half | 1.7* | 3.4* | 2.8* | 2.8* | 2.5 ± | 2.2 ± |
| life | | | | | 0.9 | 1.0 |
| (hr) | | | | | | |
| Relative | 39.5^{†} | 35.8^{†} | 25.0^{†} | 27.7^{†} | reference | reference |
| Avail. | 90.2^{††} | 81.8^{††} | 57.1^{††} | 63.3^{††} | | |
| of VX-478 | | | | | | |

a dose of 50 mg/Kg of compound 229 is equal to 40 mg/Kg of VX-478.
no compound 229 was detected in plasma at 15 min. (first data point).
*Represents the harmonic mean
^{†}Relative availability of VX-478 when compared to a prototype clinical formulation
^{††}Relative availability of VX-478 when compared to a prototype toxicology formulation

We performed a similar study on dogs using both a solid capsule formulation of compound 229 and an ethanolic/methyl cellulose solution formulation, as compared to a TPGS-containing solution formulation of VX-478. The results from this study are presented below in Table V.

TABLE V

| Compound | 229 | 229 | VX-478 |
|---|---|---|---|
| vehicle | solid | methyl | 22% |
| | capsule | cellulose | TPGS/PEG |
| | | in 5% | 400/PG |
| | | EtOH/water | |
| number of dogs | 2 | 2 | >2 |
| Molar equiv. dose/ | 17 PO | 17 PO | 17 PO |
| 478 Dose (mg/Kg) | | | |
| AUC | 16.7 ± 2.7 | 14.2 ± 3.2 | 23.5 ± 7.4 |
| (ug*hr/ml) | | | |
| Cmax (μg/ml) | 6.1 ± 1.7 | 6.3 ± 0.3 | 6.8 ± 1.1 |
| Tmax (hr) | 2.3 ± 0.6 | 0.5 ± 0.5 | 1.0 ± 0.8 |
| Relative Avail. of | 71.1 | 60.4 | reference |
| VX-478 (%) | | | |

The results demonstrate that oral administration of compound 229 as an aqueous solution resulted in improved bioavailability in comparison to the other vehicles studied, Also, following administration of compound 229, none of that compound was detected in the first time point blood sample (or later samples), suggesting first pass metabolism to VX-478. Comparison of the aqueous dose of compound 229 with the two non-aqueous formulations used for VX-478 indicated equivalence in delivery as illustrated by the range found for the bioavailability.

While we have described a number of embodiments of this invention, it is apparent that our basic constructions may be altered to provide other embodiments which utilize the products and processes of this invention. Therefore, it will be appreciated that the scope of this invention is to be defined by the appended claims, rather than by the specific embodiments which have been presented by way of example.

We claim:

1. A compound of formula I: 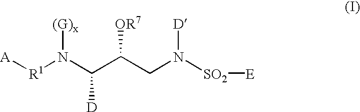
wherein:
each R¹ is independently selected from the group consisting of C(O)—, —S(O)₂—, —C(O)—C(O)—, —O—C(O)—, —O—S(O)₂, —NR²—S(O)₂—, —NR²—C(O)— and —NR²—C(O)—C(O)—;
each A is independently selected from the group consisting of 5-7 membered monocyclic heterocycles containing from 1-3 endocyclic heteroatoms, which may be optionally methylated at the point of attachment, optionally benzofused, optionally attached through a C₁-C₃ alkyl linker and optionally fused with a 5-7 membered monocyclic heterocycle containing from 1-2 endocyclic heteroatoms, and wherein unmethylated THF is expressly excluded;
each Ht is independently selected from C₃-C₇ cycloalkyl; C₅-C₇ cycloalkenyl; C₆-C₁₀ aryl; or a 5-7 membered saturated or unsaturated heterocycle, containing one or more heteroatoms selected from N, N(R²), O, S and S(O)ₙ; wherein said aryl or said heterocycle is optionally fused to Q; and wherein any member of said Ht is optionally substituted with one or more substituents independently selected from oxo, —OR², SR², —R², —N(R²)(R²), —R²—OH, —CN, —CO₂R², —C(O)—N(R²)₂, —S(O)₂—N(R²)₂, —N(R²)—C(O)—R², —C(O)—R², —S(O)ₙ—R², —OCF₃, —S(O)ₙ—Q, methylenedioxy, —N(R²)—S(O)₂(R²), halo, —CF₃, —NO₂, Q, —OQ, —OR⁷, —SR⁷, —R⁷, —N(R²)(R⁷) or —N(R⁷)₂;
each Q is independently selected from a 3-7 membered saturated, partially saturated or unsaturated carbocyclic ring system; or a 5-7 membered saturated, partially saturated or unsaturated heterocyclic ring containing one or more heteroatoms selected from O, N, S, S(O)ₙ or N(R²); wherein Q is optionally substituted with one or more groups selected from oxo, —OR², —R², —N(R²)₂, —N(R²)—C(O)—R², —R²—OH, —CN, —CO₂R², —C(O)—N(R²)₂, halo or —CF₃;
each R² is independently selected from the group consisting of H and C₁-C₃ alkyl optionally substituted with Q;
each x is independently 0 or 1;
each R³ is independently selected from the group consisting of H, Ht, C₁-C₆ alkyl and C₂-C₆ alkenyl wherein any member of said R³, except H, may be optionally substituted with one or more substituents selected from the group consisting of —OR², —C(O)—NH—R², —S(O)ₙ—N(R²)(R²), Ht, —CN, —SR², —CO₂R², NR²—C(O)—R²;
each n is independently 1 or 2;
G, when present, is selected from H, R⁷ or C₁-C₄ alkyl, or, when G is C₁-C₄ alkyl, G and R⁷ are bound to one another either directly or through a C₁-C₃ linker to form a heterocyclic ring; or
when G is not present (i.e., when x in (G)ₓ is 0), then the nitrogen to which G is attached is bound directly to the R⁷ group on —OR⁷;
each D and D′ is independently selected from the group consisting of Q; C₁-C₅ alkyl, which may be optionally substituted with one or more groups selected from C₃-C₆ cycloalkyl, —OR², —R³, —O—Q, —S—Q and Q; C₂-C₄ alkenyl, which may be optionally substituted with one or more groups selected from the group consisting of C₃-C₆ cycloalkyl, —OR², R³, O—Q and Q; C₃-C₆ cycloalkyl, which may be optionally substituted with or fused with Q; and C₅-C₆ cycloalkenyl, which may be optionally substituted with or fused with R⁶;
each E is independently selected from the group consisting of Ht; —O—Ht; Ht—Ht; —O—R³; —NR²R³; C₁-C₆ alkyl, which may be optionally substituted with one or more groups selected from the group consisting of R⁴ and Ht; and C₂-C₆ alkenyl, which may be optionally substituted with one or more groups selected from the group consisting of R⁴ and Ht; C₃-C₆ saturated carbocycle, which is optionally substituted with one or more groups selected from R⁴ or Ht; or C₅-C₆ unsaturated carbocycle, which is optionally substituted with one or more groups selected from R⁴ or Ht;
each R⁴ is independently selected from the group consisting of OR², —C(O)—NHR², S(O)₂—NHR², halo, NR²—C(O)—R² and —CN;
each R⁵ is independently selected from the group consisting of H and C₁-C₄ alkyl optionally substituted with aryl; and
each R⁶ is independently selected from the group consisting of aryl, carbocycle and heterocycle, wherein said aryl, carbocycle or heterocycle may be optionally substituted with one or more groups selected from the group consisting of oxo, —OR⁵, —R⁵, N(R⁵)(R⁵), N(R⁵)—C(O)—R⁵, —R⁵—OH, —CN, CO₂R⁵, C(O)—N(R⁵)(R⁵), halo and CF₃;
each R⁷ is independently selected from 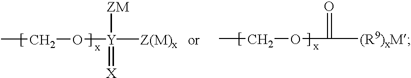
wherein each M is independently selected from H, Li, Na, K, Mg, Ca, Ba, —N(R²)₄, C₁-C₁₂-alkyl, C₂-C₁₂-alkenyl, —R⁶; wherein 1 to 4 —CH₂ radicals of the alkyl or alkenyl group, other than the —CH₂ that is bound to Z, is optionally replaced by a heteroatom group selected from O, S, S(O), S(O₂), or N(R²); and wherein any hydrogen in said alkyl, alkenyl or R⁶ is optionally replaced with a substituent selected from oxo, —OR², —R², N(R²)₂, N(R²)₃, R²OH, —CN, —CO₂R², —C(O)—N(R²)₂, S(O)₂—N(R²)₂, N(R²)—C(O)—R₂, C(O)R², —S(O)ₙ—R², OCF₃, —S(O)ₙ—R⁶, N(R²)—S(O)₂(R²), halo, —CF₃, or —NO₂;
M′ is H, C₁-C₁₂-alkyl, C₂-C₁₂-alkenyl, —R⁶; wherein 1 to 4 —CH₂ radicals of the alkyl or alkenyl group is optionally replaced by a heteroatom group selected from O, S, S(O), S(O₂), or N(R²); and wherein any hydrogen in said alkyl, alkenyl or R⁶ is optionally replaced with a substituent selected from oxo, —OR², —R², —N(R²)₂, N(R²)₃, —R²OH, —CN, —CO₂R², —C(O)—N(R²)₂, —S(O)₂—N(R²)₂, —N(R²)—C(O)—R₂, —C(O)R², —S(O)ₙ—R², —OCF₃, —S(O)ₙ—R⁶, —N(R²)—S(O)₂(R²), halo, —CF₃, or —NO₂;
Z is O, S, N(R²)₂, or, when M is absent, H;
Y is P or S;
X is O or S; and
R⁹ is C(R²)₂, O or N(R²); and wherein when Y is S, Z is not S; and
R⁶ is a 5-6 membered saturated, partially saturated or unsaturated carbocyclic or heterocyclic ring system, or an 8-10 membered saturated, partially saturated or unsaturated bicyclic ring system; wherein any of said heterocyclic ring systems contains one or more heteroatoms selected from O, N, S, S(O)ₙ or N(R²); and wherein any of said ring systems optionally contains 1 to 4 substituents independently selected from OH, C₁-C₄ alkyl, O—C₁-C₄ alkyl or OC(O)C₁-C₄ alkyl.

2. The compound according to claim 1, wherein at least one R⁷ is selected from:

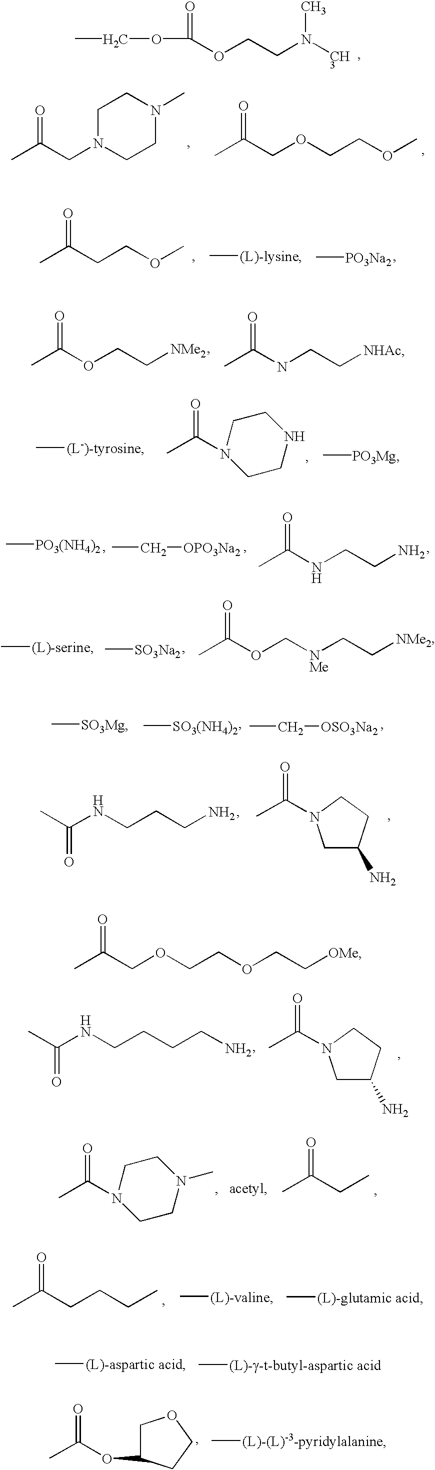

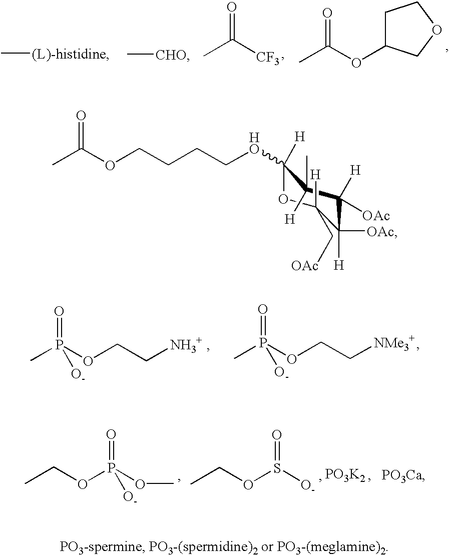

3. The compound according to claim 1, wherein D is benzyl.

4. The compound according to claim 3, wherein
A is selected from 3-(1,5-dioxane)-O—C(O)—, or 3-hydroxy-hexahydrofura[2,3]-furanyl-O—C(O)—;
D′ is (C₁-C₄)-alkyl which is optionally substituted with one or more groups selected from the group consisting of (C₃-C₆)-cycloalkyl, —OR², —R³, —O—Q and Q;
E is (C6-C₁₀)-aryl optionally substituted with one or more substituents selected from oxo, —OR², SR², —R², —N(R²)₂, —R²—OH, —CN, —C(O)O—R², —C(O)—N(R²)₂, —S(O)₂—N(R²)₂, —N(R²)—C(O)—R², —C(O)—R², —S(O)ₙ—R², —OCF₃, —S(O)ₙ—Q, methylenedioxy, —N(R²)—S(O)₂—R₂, halo, —CF₃, —NO₂, Q, —OQ, —OR⁷, —SR⁷, —R⁷, —N(R²)(R⁷) or —N(R⁷)₂; or a 5-membered heterocyclic ring containing one S and optionally containing N as an additional heteroatom, wherein said heterocyclic ring is optionally substituted with one to two groups independently selected from —CH₃, R⁴, or Ht; and
Ht, insofar as it is defined as part of R³, is defined as in claim 1 except for the exclusion of heterocycles.

5. The compound according to claim 4 wherein A is 1,3-dioxanyl.

6. The compound according to claim 5 wherein A is 1,3-dioxan-5-yl.

7. The compound according to claim 4, wherein:
G is hydrogen;
D′ is isobutyl;
E is phenyl substituted with N(R⁷)₂;
each M is independently selected from H, Li, Na, K, Mg, Ca, Ba, C₁-C₄ alkyl or —N(R²)₄; and
each M′ is H or C₁-C₄ alkyl.

8. The compound according to claim 3, wherein:
E is a 5-membered heterocyclic ring containing one S and optionally containing N as an additional heteroatom, wherein said heterocyclic ring is optionally substituted with one to two groups independently selected from —CH₃, R⁴, or Ht.

9. The compound according to claim 3, wherein:
E is Ht substituted with N(R⁷)₂;
R⁷ in the —OR⁷ group is —PO(OM)₂ or C(O)CH₂OCH₂CH₂OCH₂CH₂OCH₃ and both R⁷ in the —N(R⁷)₂ substituent of Ht are H; or R⁷ in —OR⁷ group shown in formula XXII is C(O)CH₂OCH₂CH₂OCH₃, one R⁷in the —N(R⁷)₂ substituent of Ht is C(O)CH₂OCH₂CH₂OCH₃ and the other R⁷ in the —N(R⁷)₂ substituent of Ht is H; and
wherein M is H, Li, Na, K or C₁-C₄ alkyl.

10. The compound according to claim 3, wherein R⁷ in the —OR⁷ group is —PO(OM)₂ or —C(O)—M′ and M is Na or K.

11. The compound according to claim 2, wherein:
R³ is (C₁-C₆)-alkyl, (C₂-C₆)-alkenyl, (C₅-C₆)-cycloalkyl, (C₅-C₆)-cycloalkenyl, or a 5-6 membered saturated or unsaturated heterocycle; wherein any member of R³ is optionally substituted with one or more substituents selected from the group consisting of —OR², —C(O)—NH—R², —S(O)ₙN(R²)₂, —Ht, —CN, —SR², —C(O)O—R² and N(R²)—C(O)—R²; and
D′ is (C₁-C₃)-alkyl or C₃ alkenyl; wherein D′ is optionally substituted with one or more groups selected from (C₃-C₆)-cycloalkyl, —OR², —O—Q or Q.

12. The compound according to claim 11, wherein R⁷ in the —OR⁷ group is —PO(OM)₂ or —C(O)—M′.

13. A pharmaceutical composition, comprising a compound according to any one of claims 1 to 12 in an amount effective to treat infection by a virus that is characterized by an aspartyl protease; and a pharmaceutically acceptable carrier, adjuvant or vehicle.

14. The pharmaceutical composition according to claim 13, wherein said virus is HIV.

15. The pharmaceutical composition according to claim 13, wherein said pharmaceutical composition is formulated for oral administration.

16. The pharmaceutical composition according to claim 13, further comprising one or more agents selected from an anti-viral agent, an HIV protease inhibitor other than a compound according to claim 1, and an immunostimulator.

17. The pharmaceutical composition according to claim 16, further comprising one or more agents selected from zidovudine (AZT), zalcitabine (ddC), didanosine (ddI), stavudine (d4T), 3TC, 935U83, 1592U89, 524W91, saquinavir (Ro 31-8959), L-735,524, SC-52151, ABT 538 (A80538), AG 1341, XM 412, XM 450, CPG 53,437, or tuscarasol.

18. A method for inhibiting aspartyl protease activity in a mammal, comprising the step of contacting administering to said mammal a pharmaceutical composition according to claim 13.

19. A method for treating HIV infection in a mammal comprising the step of administering to said mammal a pharmaceutical composition according to claim 13.

20. The method according to claim 19, wherein said mammal is additionally administered one or more additional agents selected from an anti-viral agent, an HIV protease inhibitor other than a compound according to claim 1, and an immunostimulator either as a part of a single dosage form with said pharmaceutical composition or as a separate dosage form.

21. The method according to claim 20, wherein said additional agent is selected from zidovudine (AZT), zalcitabine (ddC), didanosine (ddI), stavudine (d4T), 3TC, 935U83, 1592U89, 524W91, saquinavir (Ro 31-8959), L-735,524, SC-52151, ABT 538 (A80538), AG 1341, XM 412, XM 450, CPG 53,437, or tuscarasol.

22. The method according to claim 19, wherein said step of administering comprises oral administration.
